(19) United States
(12) Patent Application Publication
(10) Pub. No.: US 2012/0058068 A1
(43) Pub. Date: Mar. 8, 2012

(54) COMPOSITIONS FOR REPAIRING AND RESTORING KERATIN-CONTAINING FIBERS

(76) Inventors: Kelly van Gogh, New York, NY (US); William Onyebuagu, Chicago, IL (US)

(21) Appl. No.: 12/875,076

(22) Filed: Sep. 2, 2010

(51) Int. Cl. A61K 8/89; A61Q 5/12; A61K 8/40

(52) U.S. Cl. 424/70.9; 424/70.28; 424/70.12

(57) ABSTRACT

Disclosed are compositions that can be used as an adjunct to regular shampooing and conditioning products, wherein the disclosed compositions provide a means for repairing and restoring keratin-containing fibers to their original condition.

### FIELD

Disclosed are compositions that can be used as an adjunct to regular shampooing and conditioning products, wherein the disclosed compositions provide a means for repairing and restoring keratin-containing fibers to their original condition.

### BACKGROUND

Hair is largely composed of proteins. Artisans have described the array in which the corneocyte is present to be like a cornified envelope packed with keratin bundles. These specific keratin bundles are in a large way responsible for the stability and rigidity of the horny layer. Keratins make up about 80% of proteins present in the stratum corneum. They are structural polypeptides (40,000-80,000 Daltons) that aggregate to form filaments during the conversion of living epidermal cells into non living stratum corneum. This process is known to be catalyzed by divalent cations (i.e., zinc, calcium and magnesium) and involves interaction with filament-associated proteins fillagrin. These filaments become stabilized by cross-linking disulfide bonds formed from sulfhydryl groups that are present in the cysteine-rich keratins.

Keratin-containing fibers, for example, human hair comprises 65% to 95% by weight of proteins. These proteins are made of a structured hard α-keratin, embedded in an amorphous, proteinaceous matrix. Keratin can be damaged in a number of common ways, for example, by exposure to ultra violet radiation, mechanical means such as the wearing of a hat or by over brushing tangled and matted hair, and by chemical means such as exposure to environmental contaminants or by oxidative bleaching or coloring.

The proteins of keratin comprise a particular makeup of amino acids. The loss of a significant amount of any one particular amino acid can directly affect the fiber shaft and, therefore, its overall appearance and behavior. Ideally keratin-containing fibers would self-repair after the loss of amino acids; however, keratin is non-living tissue and thus a metabolic repair or replenishment response does not exist. In addition, several amino acids critical to the fidelity of hair cannot be synthesized by the human body, for example, lysine and methionine which is a biosynthetic precursor of cysteine the amino acid responsible for the disulfide bonds that provide shape and rigidity to hair.

Keratin-containing fibers that have been dyed are exposed to strong oxidants and can be severely damaged during the process. As a result, the fibers can become dried, brittle and have an absence of shine or body. Commercially available shampoos and conditioners are sufficient to clean keratin-containing fibers and provide a short-lived enhancement of the hair texture; however, the fibers themselves are not restored to their original composition. As such, these products tend to mask the damage done and in some instances cause to fibers to lose moisture and essential ingredients common to healthy hair.

Therefore, there is a long felt need for compositions that can be use as an adjunct conditioning agent to resupply keratin containing fibers with one or more natural ingredients that can be absorbed into the proteinaceous tissue and serve to restructure and repair damaged fibers.

### DETAILED DESCRIPTION

The materials, compounds, compositions, articles, kits and methods described herein may be understood more readily by reference to the following detailed description of specific aspects of the disclosed subject matter and the Examples included therein. Before the present materials, compounds, compositions, articles, devices, kits and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### GENERAL DEFINITIONS

In this specification and in the claims that follow, reference will be made to a number of terms, which shall be defined to have the following meanings:

All percentages, ratios and proportions herein are by weight, unless otherwise specified. All temperatures are in degrees Celsius (° C.) unless otherwise specified. All values in the disclosed TABLES are in weight/weight percentage, i.e., “a composition comprising 15% by weight” is understood to comprise 0.15 of its mass the disclosed ingredient. As such, a weight percent of a component, unless specifically stated to the contrary, is based on the total weight of the formulation or composition in which the component is included.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Admixture” or “blend” is generally used herein means a physical combination of two or more different components.

As used herein, by a “subject” is meant an individual, i.e., a human or animal having keratin-containing fiber (in general, hair) onto which subject's fibers is being applied one or more of the disclosed compositions.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “a phenylsulfamic acid” includes mixtures of two or more such phenylsulfamic acids, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “keratinous tissue,” as used herein, refers to keratin-containing layers disposed as the outermost protective covering of mammals which includes, but is not limited to, skin, hair, toenails, fingernails, cuticles, hooves, etc.

The term “topical application,” as used herein, means to apply or spread the compositions of the present invention onto the surface of the keratinous tissue.

The term “dermatologically acceptable,” as used herein, means that the compositions or components thereof so described are suitable for use in contact with human keratinous tissue without undue instability, and the like.

The term “active ingredient,” as used herein, relates to compounds that either alone or together with one or more of the disclosed ingredients is capable of modifying keratin-containing fibers or can modify or improve the application of the composition including stabilizing one or more of the other ingredients, for example, an active ingredient can be a compound that stabilizes the hydrogen peroxide or that maintains a homogeneous composition by eliminating phase separation or undesirable micelle formation. Not included in the definition of “active ingredients” are compounds that are used to dilute or aid in the dissolution of ingredients, for example, water is not an active ingredient.

The term “smoothing” and “softening” as used herein means altering the surface of the keratinous tissue such that its tactile feel is improved.

Disclosed herein are compositions useful for restoring damaged keratin-containing fibers that have been oxidatively dyed, especially human hair wherein the compositions provide a means for maintaining a durable and homogeneous color.

There is a long felt need for restoring and repairing damaged keratin-containing fibers, especially after oxidative dyeing. “Permanent” colorant compositions are not truly permanent and the process for coloring hair can damage hair fibers. Hair colorant products typically use oxidative dye color precursors what not only react with the colorant precursors, but also the protein that comprises hair, inter alia, keratin and melanin. Therefore, it is desirable to minimize any damage done to keratin-containing fibers due to unavoidable oxidative damage. In addition, the fibers can be damaged by the use of harsh shampoos and/or conditioners that provide only cleansing and masking of fiber damage respectively.

In as first aspect, disclosed herein are compositions that can restore, rebuild and replenish keratin-containing fibers that have been oxidatively dyed. These compositions can restore the condition of the epidermis layer of the fibers and overcome the effects of hard water or water that contributes to the damage of fibers when used to clean the fibers. In addition, the compositions provide a means for reducing or preventing further formation of “frizz” and increased porosity of the fibers.

The disclosed dyed keratin-containing fiber restorative compositions comprise:
A) from about 70% to about 80% by weight of a stabilized carrier system, comprising:
   a) from about 0.01% to about 5% by weight of one or more aqueous solution stabilizers or thickeners; and
   b) the balance one or more carriers;
B) from about 15% to about 35% by weight of a keratin-containing fiber conditioning and moisturizing protectant system, comprising:
   a) from about 50% to about 90% by weight of one or more active ingredients chosen from:
      i) optionally from about 25% to about 60% by weight of one or more anti-static agents;
      ii) optionally from about 5% to about 30% by weight of one or more emulsion stabilizers;
      iii) optionally from about 5% to about 90% by weight of one or more emulsifiers; and
      iv) optionally from about 1% to about 15% by weight of one or more fiber surface protection agents chosen from conditioners, wetting agents, rinse aids, emollients and mixtures thereof;
   b) from about 01% to about 25% by weight of one or more preservatives, bactericidal, bacteriostatic compounds, and mixtures thereof; and
   c) from about 0.05% to about 5% by weight of one or more chelants, pH balancers, and carriers;
C) from about 0.5% to about 10% by weight of one or more keratin-containing fiber restorative, keratin-containing fiber protective ingredients and mixtures thereof; and
D) the balance carriers and adjunct ingredients.

Shampooing is the prevalent means for cleansing keratin-containing fibers, however, shampoos, and their companion conditioners, can contribute to the damaging of dyed and non-dyed hair, especially hair that is weakened by environmental exposure, i.e., ultra violet radiation, salt water, chlorine from swimming pools, and the like. The need to feel “clean” is one major factor in the daily use of shampoos and conditions.

Another aspect of the disclosed compositions relates to compositions that can clean while restructuring and restoring keratin containing fibers without the need for harsh surfactants or other ingredients that typically comprise shampoos and conditioners. The compositions of this aspect comprise:
A) from about 0.1% to about 5% by weight of a protein restorative system, comprising:
   i) one or more essential amino acids wherein the term essential amino acid refers to amino acids that cannot be synthesized by a human; and
   ii) optionally one or more non-essential amino acids;
B) from about 1% to about 15% by weight of a keratin-containing fiber conditioning and moisturizing fiber protectant system, comprising one or more optionally present ingredients chosen from each of the following: moisturizers, wetting agents, emollients, humectants, surfactants ad conditioning agents; and
C) the balance, comprising:
   i) optionally from about 1% to about 10% by weight of the balance, one or more composition stabilizers, aesthetics, UV protectants, protein enrichment agents, chelants, antioxidants, preservatives, pH control agents, perfumes, colorants and the like; and
   ii) one or more carriers.

The disclosed compositions comprise one or more optional ingredients, however, the compositions comprise at least one ingredient from any listing of optional ingredients. In addition, any of the herein below recited ingredients can be specifically excluded from any of the disclosed compositions, for example, an individual emollient can be excluded or emollients as a class can be excluded from any of the compositions falling under the disclosure or the compositions, methods or kits recited in the appended claims.

### Ingredients

The following are non-limiting examples of the ingredients that comprise the disclosed compositions. The artisan will understand that the disclosed ingredients can function in one or more ways when formulated into a composition, for example, a compound can have surfactant properties, as well as being a humectants, emollient, or antistatic agent. As such, the categorization and/or naming of the following ingredients by function is not meant to be limiting or to exclude the dual use of any or all ingredients to provide a different function or benefit.

### Amino Acids

One category of the disclosed compositions comprises from about from about 0.1% to about 5% by weight of a protein restorative system, comprising:
a) from about 25% to about 50% by weight of a source of lysine;
b) from about 25% to about 50% by weight of a source of glycine;
c) from about 7% to about 22% by weight of a source of arginine; and
d) from about 1% to about 15% by weight of a source of cysteine.

The amino acids can be in any form, for example, the amino acid itself, a salt of the amino acid, i.e., hydrochloride or salt of an organic acid or base, as a biohydrolyzable protein, i.e., enzymatically reduced protein, hydrolyzed protein, and the like. What is meant by biohydrolyzable protein is a protein that when contacted with a keratin-containing fiber, that amino acids, especially lysine and arginine, can be taken up from the protein into the stratum corneum and become a part of the fiber itself, thereby serving to repair damage due to oxidative dyeing, mechanical, or environmental exposure.

Moisturizers

The disclosed keratin-containing repair and restorative compositions can comprise from about 5% to about 10% by weight of one or more moisturizers. In one embodiment, the compositions can comprise from about 5% to about 7% by weight of one or more moisturizers. In a further embodiment, the compositions can comprise from about 6% to about 8% by weight of one or more moisturizers. In another embodiment, the compositions can comprise from about 7% to about 10% by weight of one or more moisturizers.

The disclosed moisturizers can also comprise a moisturizing system, wherein several compatible ingredients are combined and delivered to the compositions as an admixture. For example, formulators and suppliers of moisturizing ingredients can pre-admix two or more ingredients into a stable form that can be easily formulated into a keratin-containing composition. The formulator will select these admixtures depending upon the properties of the final composition.

One example of a disclosed moisturizing system comprises:
a) from about 10% to about 90% by weight of one or more hydrating agents;
b) from about 10% to about 90% by weight of one or more polymers; and
c) from about 10% to about 90% by weight of one or more carriers.

One category of hydrating agents relates to polyhydroxy compounds, for example, is glycerin. Another category of hydrating agents relates to vitamins and other compounds which stimulate the natural hydration of keratin-containing fibers. Non-limiting examples include panthenol (vitamin B₅), milk proteins, silk proteins, and the like.

Polymers suitable for inclusion in the disclosed moisturizing system include glyceryl acrylate/acrylic acid copolymers, ethanol amine condensation copolymers as disclosed in U.S. application Ser. No. 12/281,032, included herein by reference in its entirety, and the like. Suitable carriers include water, fatty alcohols, polyhydroxy alcohols, polyethers, and the like.

One example of a category of moisturizing systems suitable for use in the disclosed compositions comprises from about 0.1% to about 1% by weight of a system, comprising:
a) from about 10% to about 90% by weight of glycerin;
b) from about 10% to about 90% by weight of glyceryl acrylate/acrylic acid copolymers; and
c) from about 10% to about 90% by weight of propylene glycol.

The above described moisturizing systems are sold as Lubrajel™ CG, Lubrajel™ DV, Lubrajel™ MS, Lubrajel™ PF, and the like available from International Specialty Products (ISP).

### Wetting Agents

The disclosed restorative hair, conditioning, and maintenance can comprise from about 0.1% to about 20% by weight of one or more silicone comprising polymers. In one embodiment, the compositions can comprise from about 0.15% to about 10% by weight of one or more silicone polymers. In another embodiment, the compositions can comprise from about 0.2% to about 8% by weight of one or more silicone polymers. In a still further embodiment, the compositions can comprise from about 0.1% to about 3% by weight of one or mere wetting agents.

Typically the silicone compounds useful herein, as a single compound, as a blend or admixture of at least two silicone compounds, or as a blend or admixture of at least one silicone compound and at least one solvent, have a viscosity of from about 1,000 to about 2,000,000 mPas at 25° C. As such, the viscosity can be measured by means of a glass capillary viscometer as set forth in Dow Corning Corporate Test Method CTM0004, Jul. 20, 1970. Non-limiting examples of suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having keratin-containing fibers restorative and/or conditioning properties can also be used.

One non-limiting example of silicone compounds that can comprise the disclosed compositions includes polyalkyl or polyaryl siloxanes having the following formula:

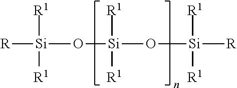

wherein R¹ is alkyl or aryl, and the index n is an integer from about 7 to about 8,000. R represents groups which block the ends of the silicone chains. The alkyl or aryl groups substituted on the siloxane chain (R¹) or at the ends of the siloxane chains R can have any structure as long as the resulting silicone remains fluid at room temperature, is dispersible, is neither irritating, toxic nor otherwise harmful when applied to the keratin-containing fibers, is compatible with the other components of the composition, is chemically stable under normal use and storage conditions, and is capable of being deposited on and conditions the keratin-containing fibers. In one embodiment, R groups include hydroxy, methyl, methoxy, ethoxy, propoxy, and aryloxy. The two R¹ groups on the silicon atom may represent the same group or different groups. In one embodiment, the two R¹ groups represent the same group. Non-limiting examples of R¹ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known to the artisan as dimethicone, and derivatives thereof, are one embodiment of the disclosed silicones. The polyalkylsiloxanes that can be used include, for example, polydimethylsiloxanes. These silicone compounds are available, for example, from the General Electric Company in their Viscasil™ and TSF 451 series, and from Dow Corning in their Dow Corning SH200 series.

The herein disclosed polyalkylsiloxanes are available, for example, as an admixture with silicone compounds having a lower viscosity. Such admixtures have a viscosity of from about 1,000 mPas to about 100,000 mPas. In another example, from about 5,000 mPas to about 50,000 mPas. Such admixtures can comprise:
i) a first silicone having a viscosity of from about 100,000 mPas to about 30,000,000 mPas at 25° C.; and
ii) a second silicone having a viscosity of from about 5 mPas to about 10,000 mPas at 25° C.

In a further example, the silicone admixture can comprise:
i) a first silicone having a viscosity of from about 100,000 mPas to about 20,000,000 mPas at 25° C.; and
ii) a second silicone having a viscosity of from about 5 mPas to about 5,000 mPas at 25° C.

Admixtures that can be included in the disclosed compositions are, for example, a blend of dimethicone having a viscosity of 18,000,000 mPas and dimethicone having a viscosity of 200 mPas available from GE Toshiba, and a blend of dimethicone having a viscosity of 18,000,000 mPas and cyclopentasiloxane available from GE Toshiba.

The silicone compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. The “silicone gums” will typically have a mass molecular weight in excess of about 200,000, generally between about 200,000 and about 1,000,000. Non-limiting examples include polydimethylsiloxane, poly(dimethylsiloxane methylvinylsiloxane) copolymer, poly(dimethylsiloxane diphenylsiloxane methylvinylsiloxane) copolymer and mixtures thereof. Silicone gums are available, for example, as an admixture with silicone compounds having a lower viscosity. Such mixtures useful herein include, for example, Gum/Cyclomethicone blend available from Shin-Etsu.

The silicone compounds that can be used include, for example, a polypropylene oxide modified polydimethylsiloxane although ethylene oxide or mixtures of ethylene oxide and propylene oxide can also be used. The ethylene oxide and polypropylene oxide level should be sufficiently low so as not to interfere with the dispersibility, wetting, or humectants properties of the silicone. These materials are also known to the artisan as dimethicone copolyols.

One example of silicones that can be used as wetting agents includes from about 0.5% to about 15% by weight of one or more composition modifiers such as PEG-12 Dimethicone sold as D.C. Surfactant™ 193C by Dow Chemical. Other wetting agents include ethyleneoxide modified polydimethyl siloxanes, for example, Silsense™ Copolyol-1 and Schercomid™ AME-100 available from Noveon Consumer Specialties and vegetable derived polyesters, for example, Pelemol™ PHS-8 available from Phoenix Chemical, Inc.

### Emollients and Humectants

The disclosed restorative hair, conditioning, and maintenance compositions can comprise from about 0.1% to about 10% by weight of one or more emollients and/or humectants.

One example of a emollient suitable for use in a disclosed conditioning composition is 2-hydroxy-N-(2-hydroxyethyl)propanamide (lactamide MEA) available as Mackamide™ LME from Mcintyre. The conditioning compositions can comprise from about 0.01% to about 5% by weight of lactamide MEA. In one embodiment, the compositions comprise from about 0.3% to about 0.7% by weight of lactamide MEA.

The disclosed compositions can comprise from about 0.1% to about 3% by weight of one or more emollients. Non-limiting examples of emollients include but are not limited to C₁₄-C₂₂ fatty alcohols, for example, cetyl alcohol, stearyl alcohol, cetearyl alcohol; C₁₄-C₂₂ fatty alcohols fatty acid esters, for example, isopropyl mysristate, isopropyl palmitate, diisopropyl dimer dilinoleate; C₈-C₁₄ fatty alcohol triglycerides, for example, caprylic/capric triglyceride; cetyl esters; C₈-C₄₀ hydrocarbons, for example, light mineral oil, white petrolatum; and waxes, for example, beeswax. Mixtures of one or more classes of emollients can also comprise the disclosed compositions, for example, a mixture of C₁₄-C₂₂ fatty alcohols and C₁₄-C₂₂ fatty alcohols fatty acid esters.

A further embodiment of emollients relates to partial esters of natural oils in combination with silicone, for example, the triglyceride extracted from Limnathes alba (Meadowfoam Seed) is partially esterified with polyethylene glycol. One example of an emollient of this embodiment includes dimethicone PEG-8 meadowfoamate.

### Surfactants

The disclosed compositions comprise from about 1% to about 3% by weight of a surfactant system. In one aspect, the compositions comprise from about 1.5% to about 2.5% by weight of a surfactant system. In another aspect, the compositions comprise from about 1% to about 2% by weight of a surfactant system. The surfactants comprising the disclosed compositions can be natural surfactants, i.e., obtained by extracting natural products, or synthetic surfactants.

### Natural Extract Surfactants

One category of suitable surfactants includes compounds that are extracted from plant material that have surfactant activity. The compositions can comprise from about 0.05% to about 0.5% by weight of one or more natural surfactants. Non-limiting examples include extracts of Gynostemma Pentapphyllum, Panax Ginseng, Sapindus mukorossi, cucumis sativus, Olea Europea, and the like. Also suitable for use are mixtures of extracts having surfactant properties.

### Anionic Surfactants

The following are non-limiting examples of anionic surfactants suitable for use in the disclosed compositions.

### 1. Alkyl Sulfates

The disclosed compositions can comprise one or more C₁₀-C₂₀ primary, branched chain and random alkyl sulfates having the formula ROSO₃M wherein R is a linear or branched chain comprising from 10 to 20 carbon atoms and M represents a water soluble cation. Non-limiting examples of alkyl sulfates suitable for use in the disclosed compositions include sodium decylsulfate, sodium dodecylsulfate, sodium tetradecylsulfate, sodium hexadecylsulfate, and sodium octadecylsulfate.

### 2. Alkyl Alkoxy Sulfates

The disclosed compositions can comprise one or more C₁₀-C₁₈ alkyl alkoxy sulfates having the formula:

CH₃(CH₂)ₓ(OCH₂CH₂)_{y}OSO₃M

wherein the index x is from 9 to 17, y is from 1 to 7 and M is a water soluble cation chosen from ammonium, lithium, sodium, potassium and mixtures thereof. A non-limiting example includes sodium dodecyl diethoxy sulfate having the formula:

CH₃(CH₂)₁₁(OCH₂CH₂)₂OSO₃Na.

Alkyl alkoxy sulfates are also commercially available as a mixture of ethoxylates, for example, sodium laureth sulfate is available as a mixture of ethoxylates, i.e., the index y is from 2 to 4. Other suitable examples include sodium laureth-2 sulfate having an average of 2 ethoxylates and a C₁₂ linear alkyl chain. Sodium laureth-2 is available as Texapon™ N 56 from Cognis Corp. Further examples of alkyl alkoxy sulfates includes sodium laureth-1 sulfate, sodium laureth-3 sulfate, sodium laureth-4 sulfate, sodium myreth-2 sulfate and sodium myreth-3 sulfate.

### 3. Alkenyl Sulfonates

The disclosed compositions can comprise one or more C₁₀-C₁₈ alkenyl sulfonates (α-olefin sulfonates) having the formula:

CH₃(CH₂)_{z}CH═CHSO₃M

wherein the index z is from 7 to 15 and M is a water soluble cation chosen from ammonium, lithium, sodium, potassium and mixtures thereof. Olefin sulfonates are commercially available as a mixture of alkenyl chains, for example, sodium C₁₄-C₁₆ olefin sulfonate Bio-Terge™ AS-40 available from Stepan. Further non-limiting examples of alkenyl sulfonates include C₁₂-C₁₆ olefin sulfonates and C₁₄-C₁₈ olefin sulfonates. Another example is C₁₂-C₁₅ pareth-15 sulfonate available as Avanel™ S 150 CG.

### 4. Alkyl Alkoxy Carboxylates

The disclosed compositions can comprise one or more C₁₀-C₁₈ alkyl alkoxy carboxylates having the formula:

CH₃(CH₂)ₓ(OCH₂CH₂)_{y}CO₂M

wherein the index x is from 9 to 17, y is from 1 to 5 and M is a water soluble cation chosen from ammonium, lithium, sodium, potassium and mixtures thereof. A non-limiting example includes sodium dodecyl diethoxy carboxylate having the formula:

CH₃(CH₂)₁₁(OCH₂CH₂)₂CO₂Na.

Alkyl alkoxy carboxylates are also commercially available as a mixture of ethoxylates, for example, sodium laureth sulfate is available as a mixture of ethoxylates, i.e., the index y is from 2 to 4. Other suitable examples include sodium laureth-2 sulfate having an average of 2 ethoxylates and a C₁₂ linear alkyl chain. Sodium laureth-2 is available as Texapon™ N 56 from Cognis Corp. Further examples of alkyl alkoxy sulfates include sodium laureth-1 sulfate, sodium laureth-3 sulfate, sodium laureth-4 sulfate, sodium myreth-2 sulfate and sodium myreth-3 sulfate.

### 5. Isethionate Esters of Alkyl Alkoxy Carboxylic Acids

The disclosed compositions can comprise one or more C₁₀-C₁₈ isethionate esters of alkyl alkoxy carboxylates having the formula:

CH₃(CH₂)ₓ(OCH₂CH₂)_{y}OCH₂C(O)OCH₂CH₂SO₃M

wherein the index x is from 9 to 17, the index y is from 1 to 5 and M is a water soluble cation. Isethionate esters of alkyl alkoxy carboxylates are described in U.S. Pat. No. 5,466,396 the disclosure of which is included herein by reference in its entirety.

### 6. Alkyl Carboxyamides

The disclosed compositions can comprise one or more C₁₀-C₁₈ alkyl carboxyamides having the formula:

CH₃(CH₂)ₓC(O)NR(CH₂)_{y}CO₂M

wherein R is hydrogen or methyl the index x is from 9 to 17, the index y is from 1 to 5 and M is a water soluble cation. A non-limiting example of an alkyl carboxyamide suitable for use in the disclosed compositions includes potassium cocoyl glycinate available as Amilite™ GCK-12 from Ajinomoto. A further example includes compounds wherein R is methyl, for example, sodium cocoyl sarcosinate.

### Zwitterionic Surfactants

The disclosed restorative hair cleansing, conditioning, and maintenance compositions can comprise from about 5% to about 25% by weight of one of more zwitterionic (amphoteric) surfactants. The following are non-limiting examples of zwitterionic surfactants suitable for use in the disclosed compositions.

### 1. Alkyl Amide Betaines

One category of zwitterionic surfactants relates to C₁₀-C₁₆ alkyl amide betaines having the formula:

CH₃(CH₂)_{w}C(O)NH(CH₂)ᵤN⁺(CH₃)₂(CH₂)ₜCO₂⁻

wherein the index w is from 9 to 15, the index u is from 1 to 5 and the index t is from 1 to 5. Non-limiting examples of betaine surfactants includes {[3-(decanoylamino)ethyl]-(dimethyl)-ammonio}acetate, {[3-(decanoylamino)ethyl](dimethyl)ammonio}-acetate, {[3-(dodecanoyl-amino)ethyl] (dimethyl)ammonio}acetate, {[3-(dodecanoylamino)propyl]-(dimethyl)-ammonio}acetate, {[3-(dodecanoylamino)-butyl] (dimethyl)ammonio}acetate, {[3-(tetra-decanoylamino)ethyl](dimethyl)-ammonio}acetate, {[3-(tertadecanoylamino)-propyl] (dimethyl)ammonio}acetate, {[3-(hexadecanoylamino)ethyl](dimethyl)-ammonio}acetate, and {[3-(hexa-decanoylamino)propyl](dimethyl)ammonio}acetate.

### 2. Alkyl Amide Sultaines

Another category of zwitterionic surfactants relates to C₁₀-C₁₆ alkyl amide sultaines having the formula:

CH₃(CH₂)_{w}C(O)NH(CH₂)ᵤN⁺(CH₃)₂(CH₂)ₜSO₃⁻

wherein the index w is from 9 to 15, the index u is from 1 to 5 and the index t is from 1 to 5. Non-limiting examples of sultaine surfactants includes {[3-(decanoylamino)ethyl]-(dimethyl)-ammonio}methanesulfonate, {[3-(decanoylamino)ethyl](dimethyl)ammonio}-methanesulfonate, {[3-(dodecanoyl-amino)ethyl](dimethyl)ammonio}methanesulfonate, {[3-(dodecanoylamino)-propyl](dimethyl)ammonio}methanesulfonate, {[3-(dodecanoyl-amino)butyl](dimethyl)-ammonio}methanesulfonate, {[3-(tetradecanoylamino)ethyl]-(dimethyl)ammonio}methane-sulfonate, {[3-(tertadecanoylamino)propyl] (dimethyl)-ammonio}methanesulfonate, {[3-(hexadecanoylamino)ethyl](dimethyl)ammonio}-methanesulfonate, and {[3-(hexadecanoylamino)propyl](dimethyl)ammonio}-methanesulfonate.

### 3. Alkyl Hydroxy Sultaines

A further category of zwitterionic surfactants relates to C₁₀-C₁₆ alkyl hydroxy sultaines having the formula:

CH₃(CH₂)_{w}N⁺(CH₃)₂CH₂CHOHCH₂SO₃⁻

wherein the index w is from 9 to 15. Non-limiting examples of alkyl hydroxy sultaine surfactants includes 3-[dodecyl(dimethyl)azaniumyl]-2-hydroxypropane-1-sulfonate (lauryl hydroxysultaine), 3-[tetradecyl(dimethyl)azaniumyl]-2-hydroxypropane-1-sulfonate (myristyl hydroxysultaine), (Z)-{dimethyl[3-(octadec9-enamido)propyl]ammonio}-methanesulfonate (oleyl hydroxysultaine), and the like.

### Nonionic Surfactants

The disclosed restorative hair cleansing, conditioning, and maintenance compositions can comprise one of more nonionic surfactants. The following are non-limiting examples of nonionic surfactants suitable for use in the disclosed compositions.

### 1. Alkyl Glucosides

One category of nonionic surfactants relates to C₈-C₁₈ alkylglycosidyl nonionic surfactant having the formula:

CH₃(CH₂)_{q}O[G]ₚH

wherein G represents a monosaccharide residue chosen from glucose, fructose, mannose, galactose, talose, allose, altrose, idose, arabinose, xylose, lyxose, ribose and mixtures thereof, the index p is from 1 to 4, the index q is from 7 to 17. The following are non-limiting examples of alkyl glucoside surfactants include (2R,3S,4S,5R,6R)-2-(hydroxymethyl)-6-octooxyoxane-3,4,5-triol (octyl glucoside, n-octyl-β-D-glucoside), (2R,3R,4S,5S,6R)-2-decoxy-6-(hydroxymethyl)tetra-hydropyran-3,4,5-triol(decyl glucoside, n-decyl-β-D-glucoside), and (2R,3R,4S,5S,6R)-2-dodecoxy-6-(hydroxymethyl)tetrahydropyran-3,4,5-triol (dodecyl glucoside, lauryl glucoside, n-dodecyl-13-D-glucoside). One example of a suitable admixture of C₈-C₁₆ alkylglycosidyl nonionic surfactants is Plantacare™ 818 UP available from Cogins Chemical Co.

### 2. Polyoxyethylene Glycol Alkyl Ethers

A further category of nonionic surfactants relates to polyoxyethylene glycol alkyl ethers having the formula:

RO(CH₂CH₂O)ₙH

wherein R is a linear or branched alkyl group having from 6 to 20 carbon atoms and n is an integer of about 2 to about 20.

On example of suitable ethoxylate alcohol surfactants are the Neodol™ ethoxylated alcohols from Shell Chemicals. Neodol™ 23-1 is a surfactant comprising a mixture of R units that are C₁₂ and C₁₃ in length with an average of 1 ethoxy unit. Non-limiting examples of ethoxylated alcohols include Neodol™ 23-1, Neodol™ 23-2, Neodol™ 23-6.5, Neodol™ 25-3, Neodol™ 25-5, Neodol™ 25-7, Neodol™ 25-9, Pluronic™ 12R3, and Pluronic™ 25R2 available from BASF.

### 3. Polyoxypropylene Glycol Alkyl Ethers

A still further category of nonionic surfactants relates to polyoxyethylene glycol alkyl ethers having the formula:

RO(CH₂CH(CH₃)O)ₙH

wherein R is a linear or branched alkyl group having from 6 to 20 carbon atoms and n is an integer of about 2 to about 20.

### 4. Polyoxyethylene Polyoxypropylene Block Copolymers

Another category of nonionic surfactants suitable for use in the disclosed compositions includes polyoxyethylene polyoxypropylene block copolymers known as “poloxamers” having the formula:

HO(CH₂CH₂)_{y1}(CH₂CH₂CH₂O)_{y2}(CH₂CH₂O)_{y3}OH

these are nonionic block copolymers composed of a polypropyleneoxy unit flanked by two polyethyleneoxy units. The indices y¹, y², and y³ have values such that the poloxamer has an average molecular weight of from about 1000 g/mol to about 20,000 g/mol. These extracellular desiccants are also well known by the trade name Pluronics™. These compounds are commonly named with the word Poloxamer followed by a number to indicate the specific co-polymer, for example Poloxamer 407 having two PEG blocks of about 101 units (y¹ and y³ each equal to 101) and a polypropylene block of about 56 units. This category of nonionic surfactant is commercially, for example, under the trade name Lutrol™ F-17 available from BASF.

### 5. Alkoxylated Alkyl Amides

A yet still further category of nonionic surfactants suitable for use in the disclosed compositions includes alkyl amides that are ethoxylate, propoxylated, or mixtures thereof, having the formula:

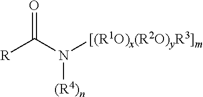

wherein R is C₇-C₂₁ linear alkyl, C₇-C₂₁ branched alkyl, C₇-C₂₁ linear alkenyl, C₇-C₂₁ branched alkenyl, and mixtures thereof. R¹ is ethylene; R² is C₃-C₄ linear alkylene, C₃-C₄ branched alkylene, and mixtures thereof; in some iterations R² is 1,2-propylene. Nonionic surfactants that comprise a mixture of R¹ and R² units can comprise from about 4 to about 12 ethylene units in combination with from about 1 to about 4 1,2-propylene units. The units can be alternating or grouped together in any combination suitable to the formulator. In one iteration, the ratio of R¹ units to R² units is from about 4:1 to about 8:1. In another iteration, a R² unit (i.e., 1,2-propylene) is attached to the nitrogen atom followed by the balance of the chain comprising from 4 to 8 ethylene units.

R³ is hydrogen, C₁-C₄ linear alkyl, C₃-C₄ branched alkyl, and mixtures thereof; preferably hydrogen or methyl, more preferably hydrogen.

R⁴ is hydrogen, C₁-C₄ linear alkyl, C₃-C₄ branched alkyl, and mixtures thereof. When the index m is equal to 2 the index n must be equal to 0 and the R⁴ unit is absent and is instead replaced by a -[(R¹O)ₓ(R²O)_{y}R³] unit.

The index m is 1 or 2, the index n is 0 or 1, provided that when m is equal to 1, n is equal to 1; and when m is 2 n is 0; in one example, m is equal to 1 and n is equal to one, resulting in one -[(R¹O)ₓ(R²O)_{y}R³] unit and R⁴ being present on the nitrogen. The index x is from 0 to about 50, in one embodiment from about 3 to about 25, in another embodiment x is from about 3 to about 10. The index y is from 0 to about 10, in one example y is 0; however, when the index y is not equal to 0, y is from 1 to about 4. In one embodiment all of the alkyleneoxy units are ethyleneoxy units.

Another category of surfactants suitable for use in the disclosed compositions include the bis(3-alkoxypropan-2-ol) sulfides, sulfoxides and sulfones as disclosed in U.S. Pat. No. 7,476,766, the disclosure of which is included herein by reference in its entirety.

### Conditioning Agents

Keratin-containing fibers can become dry and brittle due to exposure to UV radiation, chemicals used for cleaning the fibers, and from the wearing of apparel. In one embodiment, the disclosed compositions can comprise from about 0.1% to about 1.5% by weight of one or more conditioning agents.

The conditioning agents can comprise polymeric materials or small naturally occurring molecules that interact with the keratin-containing fiber to provide a benefit to the properties of the fibers. In one aspect, the disclosed compositions comprise one or more nonionic amphiphilic homopolymers or copolymers. In one embodiment, the compositions comprise polyalkylene glycols having the formula:

HO(CH₂CH₂O)ₓH

wherein the index x represent the average number of ethyleneoxy units in the glycol polymer. The index x can be represented by a whole number or a fraction. For example, a polyethylene glycol having an average molecular weight of 8,000 g/mole (PEG 8000) can be equally represented by the formulae:

HO(CH₂CH₂O)₁₈₁H or HO(CH₂CH₂O)_{181.4}H

or the polyethylene glycol can be represented by the common short hand notation: PEG 8000. This notation, common to the artisan is used interchangeably throughout the specification to indicate polyethylene glycols and their average molecular weight. The formulator will understand that depending upon the source of the polyethylene glycol, the range of molecular weights found within a particular sample or lot can range over more or less values of x. For example, one source of PEG 8000 can include polymers wherein the value of x can be from about 175 to about 187, whereas another source can report the range of molecular weights such that x can be from about 177 to about 184. In fact, the formulator, depending upon the condition of the user's keratin-containing fibers, can provide a conditioner system that comprises an admixture of different polyethylene glycols in varying amounts. For example, from about 0.5% by weight of the restorative cleansing or conditioning system can comprise PEG 4000 and 0.5% by weight of the restorative cleansing or conditioning system can comprise PEG 8000.

One non-limiting example of a suitable conditioning agent includes polyethylene glycols having an average molecular weight from about 1200 g/mol to about 20,000 g/mol. A further example of a suitable conditioning agent includes the polyethylene glycols having an average molecular weight from about 3,000 g/mol to about 12,000 g/mol. Another example of a suitable conditioning agent includes the polyethylene glycols having an average molecular weight from about 4,000 g/mol to about 10,000 g/mol. One non-limiting example of a suitable conditioning agent is a polyethylene glycol having an average molecular weight of about 8,000 g/mol, for example, PEG 8000.

Another embodiment of conditioning agents relates to polypropylene glycols having the formula:

HO[CH(CH₃)CH₂O]ₓH

wherein the index x represent the average number of propyleneoxy units in the glycol polymer. As in the case of ethylene glycols, for propylene glycols the index x can be represented by a whole number or a fraction. For example, a polypropylene glycol having an average molecular weight of 8,000 g/mole (PEG 8000) can be equally represented by the formulae:

HO[CH(CH₃)CH₂O]₁₃₈H or HO[CH(CH₃)CH₂O]_{137.6}H

or the polypropylene glycol can be represented by the common, short hand notation: PPG 8000.

One non-limiting example of the disclosed conditioning agents includes polypropylene glycols having an average molecular weight from about 1200 g/mol to about 20,000 g/mol. A further example of the disclosed conditioning agents includes the polypropylene glycols having an average molecular weight from about 3,000 g/mol to about 12,000 g/mol. Another example of the disclosed conditioning agents includes the polypropylene glycols having an average molecular weight from about 4,000 g/mol to about 10,000 g/mol. One non-limiting example of the disclosed conditioning agents includes a polypropylene glycol having an average molecular weight of about 8,000 g/mol, for example, PER 8000.

Polypropylene glycols can be admixed with polyethylene glycols to form a conditioning agent system for use in the disclosed restorative cleansing or conditioning systems.

A further example of conditioning agents includes poloxamers having the formula:

HO(CH₂CH₂)_{y1}(CH₂CH₂CH₂O)_{y2}(CH₂CH₂O)_{y3}OH

these are nonionic block copolymers composed of a polypropyleneoxy unit flanked by two polyethyleneoxy units. The indices y¹, y², and y³ have values such that the poloxamer has an average molecular weight of from about 1000 g/mol to about 20,000 g/mol. These nonionic conditioning agents are also well known by the trade name Pluronics™ These compounds are commonly named with the word Poloxamer followed by a number to indicate the specific co-polymer, for example Poloxamer 407 having two PEG blocks of about 101 units (y¹ and y³ each equal to 101) and a polypropylene block of about 56 units. This extracellular desiccant is available from BASF under the trade name Lutrol™ F-17.

One iteration of this embodiment relates to restorative cleansing or conditioning systems that comprise form about 0.1% to about 1.0% by weight of poloxamer 185 [CAS Reg. No. 9003-11-6] wherein the indices y¹, y², and y³ have the average values of 19, 30 and 10 respectively.

In another aspect, the disclosed compositions can comprise a restorative cleansing or conditioning system comprising lysine as a conditioning agent. The disclosed compositions can comprise from about 0.0001% to about 1% by weight of lysine. In one aspect, the compositions comprise from about 0.005% to about 0.5% by weight of lysine.
i) from about 0.005% to about 0.5% by weight of lysine; and
ii) from about 0.1% to about 1.0% by weight of poloxamer 185.

### Emulsifiers

The disclosed compositions can further comprise from about 5% to about 25% by weight of one or more emulsifiers.

Non-limiting examples of emulsifiers include C₁₄-C₂₂ fatty alcohols chosen from 1-tetradecanol (myristyl alcohol), 1-hexadecanol (cetyl alcohol), cis-9-hexadecen-1-ol (palmitoleyl alcohol), 1-octadecanol (stearyl alcohol), cis-9-octadecen-1-ol (oleyl alcohol), trans-9-octadecen-1-ol (elaidyl alcohol), 1-eicosanol (arachidyl alcohol), and 1-docosanol (behenyl alcohol). Further non-limiting examples of emulsifiers include esters of C₁₄-C₂₂ fatty alcohols and inorganic acids chosen from di-1-tetradecanyl phosphate (di-myristyl phosphate), di-1-hexadecanyl phosphate (di-cetyl phosphate), di-cis-9-hexadecen-1-yl phosphate (di-palmitoleyl phosphate), di-1-octadecanyl phosphate (di-stearyl phosphate), di-cis-9-octadecen-1-yl phosphate (di-oleyl phosphate), di-trans-9-octadecen-1-yl phosphate (di-elaidyl phosphate), di-1-eicosanyl phosphate (di-arachidyl phosphate), di-1-docosanyl phosphate (di-behenyl phosphate), 1-tetradecanyl sulfate (myristyl sulfate), 1-hexadecanyl sulfate (cetyl sulfate), cis-9-hexadecen-1-yl sulfate (palmitoleyl sulfate), 1-octadecanyl sulfate (stearyl sulfate), cis-9-octadecen-1-yl sulfate (oleyl sulfate), trans-9-octadecen-1-yl sulfate (elaidyl sulfate), 1-eicosanyl sulfate (arachidyl sulfate), and 1-docosanyl sulfate (behenyl sulfate).

Yet further non-limiting examples of emulsifiers include ethers of polyoxyethylene, polyoxypropylene, and polyoxyethylene/polyoxypropylene and inorganic acids or ethers of C₁₄-C₂₂ fatty alcohols chosen from PPG-5-Ceteth-20 phosphate, Ceteth-10 phosphate, Ceteth-10, and Ceteareth-20. Still further non-limiting examples of emulsifiers includes

non-ionic surfactants, for example, polysorbate 60, sorbitan monostearate, polyglyceryl-4 oleate, polyoxyethylene(4) lauryl ether, and the like. In certain embodiments, the emulsifier is chosen from poloxamers (e.g., Pluronic™ F68, also known as Poloxamer™ 188, a poly(ethylene glycol)-block-poly(propylene glycol)-block-poly(ethylene glycol), available from BASF, Ludwigshafen, Germany) and sorbitan trioleate (e.g., Span™ 85 available from Uniqema, New Castle, Del.).

### Composition Stabilizers and Adjunct Ingredients

In addition to the disclosed moisturizers, emollients, humectants, surfactants, and conditioning agents, the disclosed compositions can further comprise one or more composition stabilizers and adjunct ingredients. Non-limiting examples of adjunct ingredients include foaming agents emulsifiers viscosity control agents thickening polymers antistatic agents, detangling agents UV protectants, chelating agents, anti-oxidation agents, antibacterial agents, preservatives, pH adjusters, shine agents, solubilizers, fragrances, colorants, pearlizers, and the like.

Included within adjunct ingredients are carriers. The disclosed compositions can comprise from about 50% to about 90% by weight of one or more carriers. In one embodiment, the compositions comprise from about 65% to about 80% by weight of one or more carriers. In one aspect, water is the sole carrier. In a further aspect water comprises from about 80% to about 99% by weight of the carrier. In a still further aspect water comprises from about 90% to about 95% by weight of the carrier. The carrier can also comprise one or more alcohols. Non-limiting examples of suitable alcohols include ethanol, n-propanol, iso-propanol, and propanediol. The formulator can, however, choose any mixture of organic solvents and/or water in combination which affords a stabile and homogeneous solution.

### Foaming Agents

The disclosed restorative hair cleansing, conditioning, and maintenance compositions can comprise from about 0.5% to about 10% by weight of one or more foaming agents. Foaming agents are also referred to as structurants by the artisan although the compounds themselves can have surfactant properties, for example, the disclosed foaming agents can be considered by the artisan in some embodiments to be nonionic surfactants. The following are non-limiting examples of categories of foaming agents:
i) alkanolamides, for example, cocamide MEA, cocamide DEA and Cocamide MIPA;
ii) C₈-C₂₄ linear or branched fatty acids, for example, dodecanoic acid (lauric acid), tetradecanoic acid (myristic acid), and the like;
iii) C₈-C₂₄ linear or branched fatty acid esters, for example, methyl dodecanoate (methyl laurate), methyl tetradecanoate (methyl myristate), and the like;
iv) C₁₀-C₂₄ linear alcohols or C₁₀-C₂₄ ethoxylated and/or propoxylated linear alcohols, for example, dodecanol, tetradecanol, laureth-2, laureth-3, laureth-4, and the like.

In one aspect the compositions comprise from about 1.5% to about 5% by weight of a C₁₀-C₁₆ alkyl monoethanolamine Non-limiting examples of C₁₀-C₁₆ alkyl monoethanolamines include N-(2-hydroxyethyl)decanamide (C₁₀ alkyl monoethanolamine), N-(2-hydroxyethyl)-dodecanamide (C₁₂ alkyl monoethanolamine, cocamide MEA), and N-(2-hydroxyethyl)tetra-decanamide (C₁₆ alkyl monoethanolamine).

In another aspect the compositions comprise from about 1.5% to about 5% by weight of a C₁₀-C₁₆ alkyl diethanolamine. Non-limiting examples of C₁₀-C₁₆ alkyl diethanolamines include N,N-bis(2-hydroxyethyl)decanamide (C₁₀ alkyl diethanolamine), N,N-bis(2-hydroxyethyl)-dodecanamide (C₁₂ alkyl diethanolamine, cocamide DEA), and N,N-bis(2-hydroxyethyl)tetra-decanamide (C₁₋₆ alkyl diethanolamine).

### Viscosity Control Agents

The disclosed compositions can comprises from about 2% to about 7% by weight of one or more viscosity control agents. The formulations of the present invention can also comprise a viscosity-enhancing agent. Examples of suitable viscosity enhancing agents include long chain alcohols, for example, cetyl alcohol, stearyl alcohol, cetearyl alcohol; cellulose ethers such as hydroxypropylmethylcellulose, hydroxyethylcellulose, hydroxypropylcellulose, and carboxymethylcellulose; polysaccharide gums such as xanthan gum; and homopolymers and copolymers of acrylic acid crosslinked with allyl sucrose or allyl pentaerythriol such as those polymers designated as carbomers in the United States Pharmacopoeia. Suitable carbomers include, for example, those available as Carbopol™ 934P, Carbopol™ 971P, Carbopol™ 940, Carbopol™ 974P, Carbopol™ 980, and Pemulen™ TR-1 (USP/NF Monograph; Carbomer 1342), all available from Noveon, Cleveland, Ohio.

The disclosed activator compositions can comprise from about 0.5% to about 5% by weight of one or more viscosity control agents. On category of suitable viscosity control agents are the copolymers of itaconate monoesters having the formula:

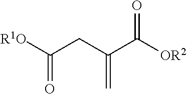

wherein one of R¹ and R² are hydrogen and the other of R¹ and R² is —(CH₂CH₂O)ₓR³, wherein x is an integer from about 1 to about 100. In one embodiment useful for the disclosed activator compositions, the index x is from about 10 to about 40, whereas in a further embodiment x is about 20. R³ is C₈-C₃₀ alkyl, in one embodiment R³ is C₁₂-C₂₀ alkyl. Non-limiting examples of viscosity control agents comprising an itaconate monomer includes acrylic acid or methacrylic acid/itaconic acid polyethoxyalkyl ester copolymers (INCI name: acrylates/Steareth-20 itaconate copolymer and acrylates/Ceteth-20 itaconate copolymer)), such as marketed by the firm National Starch, U.S.A. under the trademark Structure™ 2001 and Structure™ 3001, for example, acrylates/steareth-20 itaconate copolymer, acrylates/ceteth-20 itaconate methacrylates/steareth-20 itaconate copolymer, methacrylates/ceteth-20 itaconate acrylates/steareth-20 itaconate copolymer and acrylates/ceteth-20 itaconate copolymer.

The compositions can contain one or more thickeners that assist in maintaining an increased viscosity of the final composition. Suitable thickeners are those set forth above with respect to the oxidative composition, and in the same ranges. Also suitable are a variety of water soluble anionic thickening polymers such as those disclosed in U.S. Pat. No. 4,240,450, which is hereby incorporated by reference in its entirety. In one aspect, the activator compositions comprise from about 0.01% to about 5% by weight of a thickener useful as a viscosity control agent. In one embodiment the activator composition comprises from about 0.05% to about 4% by weight of a thickener useful as a viscosity control agent. In a further embodiment the activator composition comprises from about 0.1% to about 3% by weight of a thickener useful as a viscosity control agent. Examples of such anionic polymers are copolymers of vinyl acetate and crotonic acid, graft copolymers of vinyl esters or acrylic or methacrylic acid esters, cross-linked graft copolymers resulting from the polymerization of at least one monomer of the ionic type, at least one monomer of the nonionic type, polyethylene glycol, and a crosslinking agent, and the like. Preferred are acrylate copolymers such as steareth-10 alkyl ether acrylate copolymer.

### Thickening Polymer

The disclosed restorative hair cleansing, conditioning, and maintenance compositions can comprise from about 0.05% to about 5% by weight of one or more thickeners. In one embodiment, the compositions comprise from about 0.05% to about 2% by weight of one or more thickeners. In another embodiment, the compositions comprise from about 0.1% to about 0.5% by weight of one or more thickeners.

In general, one category of thickening polymers useful herein are those which can provide the composition with an appropriate viscosity of from about 1,000 cps to about 50,000 cps. In another embodiment, the viscosity is from about 5,000 cps to about 40,000 cps. In a further embodiment, the viscosity is from about 10,000 cps to about 30,000 cps. The viscosity is conveniently measured at 25° C. utilizing a Brookfield Viscometer at shear rate of 1.0 rpm.

A variety of thickening polymers can be used in the disclosed compositions. Non-limiting examples of thickening polymers useful herein include, for example, cellulose and its derivatives such as cellulose ethers, hydrophobically modified cellulose ethers, and quaternized celluloses; nonionic guar gums; cationic guar gums; crosslinked polymers such as nonionic crosslinked polymers and cationic crosslinked polymers; and acrylate polymers such as sodium polyacrylate, polyethylacrylate, and polyacrylamide. The thickening polymers useful herein may include the polymers disclosed below under the title “CATIONIC POLYMER”. Among a variety of thickening polymers, preferred are nonionic guar gums.

A cationic guar polymer useful herein can have a molecular weight of from about 100,000 AMU (Atomic Mass Unit) to about 4,000,000 AMU. In another embodiment, a cationic guar polymer useful herein can have a molecular weight of from about 500,000 AMU to about 3,500,000 AMU. In a further embodiment, a cationic guar polymer useful herein can have a molecular weight of from about 1,000,000 AMU to about 3,000,000 AMU. In a yet further embodiment, a cationic guar polymer useful herein can have a molecular weight of from about 1,600,000 AMU to about 2,800,000 AMU. One example of a commercially available cationic thickener is Jaguar™ C-14S available from Rhodia.

A nonionic guar polymer useful herein can have a molecular weight of from about 100,000 AMU (Atomic Mass Unit) to about 4,000,000 AMU. In another embodiment, a nonionic guar polymer useful herein can have a molecular weight of from about 500,000 AMU to about 3,500,000 AMU. In a further embodiment, a nonionic guar polymer useful herein can have a molecular weight of from about 1,000,000 AMU to about 3,000,000 AMU. In a yet further embodiment, a nonionic guar polymer useful herein can have a molecular weight of from about 1,600,000 AMU to about 2,800,000 AMU. One example of a commercially available cationic thickener is Jaguar™ HP-105 available from Rhodia.

Further examples of useful thickening agents include, but are not limited to, any of the various cellulosic ethers known in the art, for example, a hydroxyethyl cellulose thickener such as Cellosize™ PCG-10 available from Dow Chemical. In addition, a hydroxyethyl cellulose sodium acetate can be used, for example Cellosize™ 52000H available from Dow Chemical. Further examples of thickeners that can be used include carboxypolymethylene, polyethylene oxide, polyacrylic acids, copolymers of polyacrylic acid, polyacrylates, polyacrylamides, copolymers of polyacrylic acid and polyacrylamide, polyvinylpyrrolidone (PVP), PVP-vinyl acetate copolymers, carboxymethylcellulose, carboxypropylcellulose, polysaccharide gums, and proteins.

The one or more thickening agents may be added in order to alter the rheology or consistency of the composition so that it can be applied more evenly, while reducing or eliminating waste. The thickening agent can increase the viscosity of the composition and results in a composition having the consistency of a cream or lotion that more easily stays where placed. In the absence of a thickening agent, the hair restorative composition is more fluid, having a consistency more similar to water, which can make it difficult to apply evenly throughout the damaged hair without requiring a large excess. Applying a large excess of particularly as the composition begins to stiffen or solidify during the hair restoration process.

### Antistatic Agents

The disclosed restorative hair, conditioning, and maintenance compositions can comprise from about 0.05% to about 5% by weight of one or more antistatic agents. In one embodiment, the compositions comprise from about 0.05% to about 2% by weight of one or more antistatic agents. In another embodiment, the compositions comprise from about 0.1% to about 0.5% by weight of one or more antistatic agents.

One example of an antistatic agent that can be used to provide treated keratin-containing fibers with non-clumping or non-tangling properties making wet and dry combing easier are the quaternary cellulose polymers wherein their quaternary structure contains C₁₀-C₂₂ linear alkyl units. Non-limiting examples include PG-hydroxyethyl cellulose laurdiammonium chloride (laurdimonium hydroxyethylcellulose, Crodacel™ QL) available from Croda, PG-hydroxyethyl cellulose cocodiammonium chloride (cocodimonium hydroxyethylcellulose, Crodacel™ QL) available from Croda, and PG-hydroxyethyl cellulose steardiammonium chloride (steardimonium hydroxyethylcellulose, Crodacel™ QL) available from Croda, each having an average molecular weight of approximately 100,000 AMU. These antistatic agents are characterized as having an average of 1.2 moles of alkyl quaternary groups per anhydro glucose units.

Further non-limiting examples of synthetic or nonsynthetic cationic polymers that can act as antistatic conditioning agents are polyquaternium agents, for example, polyquaternium-1, polyquaternium-2, polyquaternium-4, polyquaternium-5, polyquaternium-6 (also known as Merquat™ 1000 available from Nalco), polyquaternium-7 (also known as Merquat™ 5500 available from Nalco), polyquaternium-8, polyquaternium-9, polyquaternium-10 (also known as Polymer JR™ 400, sold by Amerchol), polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22 (also known as Merquat™ 280, 281 and 298 available from Nalco), polyquaternium-24, polyquaternium-27, polyquaternium-28, polyquaternium-29 (also known as Kytamer™ KCO available from Amerchol), polyquaternium-30, polyquaternium-31, polyquaternium-32, polyquaternium-33, polyquaternium-34, polyquaternium-35, polyquaternium-36, polyquaternium-37, polyquaternium-39 (also known as Merquat™ 3300 and 3331 available from Nalco), polyquaternium-44, polyquaternium-27 (also known as Merquat™ 2001 available from Nalco) and polyquaternium-55.

### Detangling Agent

The disclosed restorative cleansing or conditioning compositions can comprise one or more detangling agents chosen from C₁₀-C₂₂ alkyl esters of alkoxylated trimethylolpropane, C₁₀-C₂₂ alkenyl mono-ene esters of alkoxylated trimethylolpropane, C₁₀-C₂₂ alkynyl mono-yne esters of alkoxylated trimethylolpropane, and mixtures thereof can have the formula:

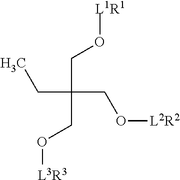

wherein R¹, R² and R³ are each independently chosen from:
i) hydrogen;
ii) C₁₀-C₂₂ carboxyalkyl;
iii) C₁₀-C₂₂ carboxyalkenyl; and
iv) C₁₀-C₂₂ carboxyalkynyl;
   L¹, L² and L³ are linking units each independently chosen from:
i) —(CR^{4a}R^{4b})_{w}O—; and
ii) —[(CR^{5a}R^{5b})_{w}]ₓ[O(CR^{6a}R^{6b})_{y}]_{z}O—;

the index w is from 2 to 150, the index x is from 1 to 50, the index y is from 3 to 300, the index z is from 1 to 20; and

R⁴a, R^{4b}, R^{5a}, R^{5b}, R^{6a}, and R^{6b} are each independently chosen from:
i) hydrogen; and
ii) methyl.

The R¹, R² and R³ units comprise C₁₀-C₂₂ fatty acid residues of the corresponding C₁₀-C₂₂ fatty acids. In a one aspect of the disclosed detangling agents, R¹, R² and R³ are from 90% to about 99.9% by weight of a single C₁₀-C₂₂ carboxyalkenyl units. Non-limiting examples of R¹, R² and R³ according to this aspect include the following cis-D carboxyalkenyl residues:
i) —C(O)(CH)₇CH═CH(CH₂)₃CH₃ (myristoleoyl);
ii) —C(O)(CH)₇CH═CH(CH₂)₅CH₃ (palmitoleoyl);
iii) —C(O)(CH)₄CH═CH(CH₂)₈CH₃ (sapienoyl);
iv) —C(O)(CH)₇CH═CH(CH₂)₇CH₃ (oleoyl);
v) —C(O)(CH)₇CH═CHCH₂(CH₂)₄CH₃ (linoleoyl); and
   vi) —C(O)(CH)₁₁CH═CH(CH₂)₇CH₃ (erucoyl).

In another aspect of the disclosed detangling agents, R¹, R² and R³ are from 90% to about 99.9% by weight of a mixture of C₁₀-C₂₂ carboxyalkenyl units. Non-limiting examples of R¹, R² and R³ according to this aspect include the following cis-D carboxyalkenyl residues:
i) —C(O)(CH)₇CH═CH(CH₂)₃CH₃ (myristoleoyl);
ii) —C(O)(CH)₇CH═CH(CH₂)₅CH₃ (palmitoleoyl);
iii) —C(O)(CH)₄CH═CH(CH₂)₈CH₃ (sapienoyl);
iv) —C(O)(CH)₇CH═CH(CH₂)₇CH₃ (oleoyl);
v) —C(O)(CH)₇CH═CHCH₂(CH₂)₄CH₃ (linoleoyl); and
vi) —C(O)(CH)₁₁CH═CH(CH₂)₇CH₃ (erucoyl).

The first aspect of L¹, L² and L³ relates to linking units that comprise polyoxyethylene polyoxypropylene residues that are derived from the random co-polymerization of ethylene oxide and propylene oxide. The L¹, L² and L³ units can be attached to the central trimethylolpropane core by either grafting the L¹, L² and L³ units onto the trimethylolpropane core or by reacting a polyoxyethylene polyoxypropylene precursor with the trimethylolpropane core.

A one embodiment of this aspect includes a detangling agent wherein each of L¹, L² and L³ comprises greater than about 90% by weight of a polyoxyethylene polyoxypropylene residue comprising about 120 polyoxyethylene and 10 polyoxypropylene units (PEF/PPG 120/10) represented by the formula:

—[(CH₂CH₂)]₁₂₀[O(CH₂CH₂)₃]₁₀O—.

Non-limiting examples of detangling agents according to this embodiment include PEG/PPG-120/10 trimethylolpropane trioleate (and) laueth-2 [CAS Reg. No. 37339-03-0] available as an admixture with ethoxylated lauryl alcohol from Cognis as Arylpon™ TT.

### UV Radiation Absorbers

The disclosed compositions can comprise from about 0.005% to 0.05% by weight of one or more UV absorbers. This includes the disclosed restorative cleansing or conditioning systems, as well as the disclosed activator compositions. In one aspect the compositions comprise from about 0.01% to about 0.04% by weight of one or more UV absorbers. The following are non-limiting examples of UV absorbers ethylhexyl methoxycinnamate (octyl methoxycinnamate), methoxydibenzoylmethane (i.e., Parsol™ 1789, Eusolex™ 9020 and Escalol™ 517), polyoxypropylene, polyoxyethylene ethers of aliphatic alcohols (for example, PPG-26 Buteth-26), ethylhexyl salicylate, and polyoxyethylene derivatives of hydroxy fatty acid containing fats and oils, (for example, PEG-40 hydrogenated castor oil). One UV protectant suitable for use is Coabsorb™ EW which comprises an admixture of ethylhexyl methoxycinnamate, butyl methoxydibenzoyl methane, PPG-26-buteth-26, ethylhexyl salicylate, PEG-40-hydrogenated castor oil available from Sensient/LCW.

The following are further non-limiting examples of UV protectants suitable for use in the disclosed compositions. Aminobenzoic Acid, Cinoxate, Diethanolamine Methoxy-cinnamate, Digalloyl Trioleate, Dioxybenzone, Ethyl 4-[bis(Hydroxypropyl)]Aminobenzoate, Glyceryl Aminobenzoate, Homosalate, Lawsone with Dihydroxyacetone, Menthyl Anthranilate, Octocrylene, Octyl Methoxycinnamate, Octyl Salicylate, Oxybenzone, Padimate O, Phenylbenzimidazole Sulfonic Acid, Red Petrolatum, Sulisobenzone, Titanium Dioxide, and Trolamine Salicylate, cetaminosalol, Allatoin PABA, Benzalphthalide, Benzophenone, Benzophenone 1-12, 3-Benzylidene Camphor, Benzylidenecamphor Hydrolyzed Collagen Sulfonamide, Benzylidene Camphor Sulfonic Acid, Benzyl Salicylate, Bornelone, Bumetriozole, Butyl Methoxydibenzoyl-methane, Butyl PABA, Ceria/Silica, Ceria/Silica Talc, Cinoxate, DEA-Methoxycinnamate, Dibenzoxazol Naphthalene, Di-t-Butyl Hydroxybenzylidene Camphor, Digalloyl Trioleate, Diisopropyl Methyl Cinnamate, Dimethyl PABA Ethyl Cetearyldimonium Tosylate, Dioctyl Butamido Triazone, Diphenyl Carbomethoxy Acetoxy Naphthopyran, Disodium Bisethylphenyl Tiamminotriazine Stilbenedisulfonate, Disodium Distyrylbiphenyl Triaminotriazine Stilbenedisulfonate, Disodium Distyrylbiphenyl Disulfonate, Drometrizole, Drometrizole Trisiloxane, Ethyl Dihydroxypropyl PABA, Ethyl Diisopropylcinnamate, Ethyl Methoxycinnamate, Ethyl PABA, Ethyl Urocanate, Etrocrylene Ferulic Acid, Glyceryl Octanoate Dimethoxycinnamate, Glyceryl PABA, Glycol Salicylate, Homosalate, Isoamyl p-Methoxycinnamate, Isopropylbenzyl Salicylate, Isopropyl Dibenzolylmethane, Isopropyl Methoxycinnamate, Menthyl Anthranilate, Menthyl Salicylate, 4-Methylbenzylidene, Camphor, Octocrylene, Octrizole, Octyl Dimethyl PABA, Octyl Methoxycinnamate, Octyl Salicylate, Octyl Triazone, PABA, PEG-25 PABA, Pentyl Dimethyl PABA, Phenylbenzimidazole Sulfonic Acid, Polyacrylamidomethyl Benzylidene Camphor, Potassium Methoxycinnamate, Potassium Phenylbenzimidazole Sulfonate, Red Petrolatum, Sodium Phenylbenzimidazole Sulfonate, Sodium Urocanate, TEA-Phenylbenzimidazole Sulfonate, TEA-Salicylate, Terephthalylidene Dicamphor Sulfonic Acid, Titanium Dioxide, Zinc Dioxide, Serium Dioxide, TriPABA Panthenol, Urocanic Acid, and VA/Crotonates/Methacryloxy-benzophenone-1 Copolymer.

### Metal Chelants

Because the source of oxidizer, for example, hydrogen peroxide in the disclosed composition are susceptible to degradation in the presence of metals, the compositions can comprise one or more compatible metal chelants. Organic acids, for example, citric acid can be used as a metal chelant. The use of organic acids can have the added benefit as acting as a part of a buffer system to regulate the pH of the activator composition. Non-limiting examples of organic acids includes malonic acid, succinic acid, adipic acid fumaric acid, malic acid, maleic acid, citric acid, ethylenediamine tertraacetic acid, N-(hydroxyethyl)-ethylenediaminetriacetic acid, and the like.

Another category of chelants are those disclosed in U.S. Pat. No. 5,747,440, U.S. Pat. No. 4,138,478, U.S. Pat. No. 3,202,579, U.S. Pat. No. 3,542,918, U.S. Pat. No. 5,693,854, U.S. Pat. No. 5,472,642, U.S. Pat. No. 4,983,315, and U.S. Pat. No. 5,284,972 each of which is incorporated herein in their entirety. Exemplary diamine dipolyacids suitable for use herein include ethylenediamine-N,N′-disuccinic acid (EDDS), ethylenediamine-N,N′-diglutaric acid (EDDG), 2-hydroxypropylenediamine-N,N′-disuccinic acid (HPDDS), all disclosed in European Patent EP0687292, ethylenedicysteic acid (EDC) disclosed in U.S. Pat. No. 5,693,854, diaminoalkyldi(sulfosuccinic acids) (DDS) disclosed in U.S. Pat. No. 5,472,642 and EDDHA (ethylenediamine-N,N′-bis(ortho-hydroxyphenyl acetic acid)), a method of preparation of which is disclosed in EP331556. A preferred monoamine monoamide-N,N′-dipolyacid is glycinamide-N,N′-disuccinic acid (GADS), described in U.S. Pat. No. 4,983,315.

In one example sodium stannate is used as a chelant.

### pH Modifiers or Adjusters

The disclosed compositions can comprise from about 0.01% to about 1% by weight of one or more pH modifiers. A non-limiting examples of suitable pH adjusters include citric acid, malonic acid, maleic acid, sodium phosphate, disodium phosphate, triethanolamine, diethanolamine, and the like.

In addition to the above disclosed ingredients, the disclosed compositions can further the following adjunct ingredients:
i) pearlizers, for example, Mica(and) CI-77891(and)Triethoxycaprylylsilane sold as Covapearl™ Silver 939AS available from Sensient/LCW;
ii) amino acid comprising adjunct ingredients comprising admixtures of amino acids, for example, Prodew™ available from Ajinomoto,
iii) keratin-containing fiber benefit ingredients, for example, Helianthus Annuus (Sunflower) Seed Oil and Caviar Extract sold as Lipidami™ Caviar available from Alban Muller,
iv) one or more opacifiers, for example glycol distearate sold as Mackester™ EGDS available from McIntyre Chemicals.

### Compositions for Restoring

### Damaged Keratin-Containing Fibers

The damaged fiber restorative compositions disclosed herein are suitable for us on keratin-containing fibers, especially human hair that has been damaged due to mechanical and environmental exposure, as well as damage caused by hair treatments, inter alia, coloring, caustic cleansers, hair spray, and the like. Damaged fibers become broken and with the ends especially shredded and split thereby exposing the underlying fiber core to further damage.

One aspect of the disclosed compositions comprise an admixture of protein-based ingredients to replenish the whole hair fiber internally while providing an effective barrier to environmental damage, inter alia, UV damage, chemical damage from water borne chloride ions, and from the caustic effects of shampoos and conditioners. The compositions comprise:
A) from about 70% to about 80% by weight of a stabilized carrier system, comprising:
   a) from about 0.01% to about 5% by weight of one or more aqueous solution stabilizers or thickeners; and
   b) the balance one or more carriers;
B) from about 15% to about 35% by weight of a keratin-containing fiber conditioning and moisturizing protectant system, comprising:
   a) from about 50% to about 90% by weight of one or more active ingredients chosen from:
      i) from about 25% to about 60% by weight of one or more anti-static agents;
      ii) from about 5% to about 30% by weight of one or more emulsion stabilizers;
      iii) from about 5% to about 90% by weight of one or more emulsifiers; and
      iv) from about 1% to about 15% by weight of one or more fiber surface protection agents chosen from conditioners, wetting agents, rinse aids, emollients and mixtures thereof;
   b) from about 01% to about 25% by weight of one or more preservatives, bactericidal, bacteriostatic compounds, and mixtures thereof; and
   c) from about 0.05% to about 5% by weight of one or more chelants, pH balancers, and carriers;
C) from about 0.5% to about 10% by weight of one or more keratin-containing fiber restorative, keratin-containing fiber protective ingredients and mixtures thereof; and
D) the balance carriers and adjunct ingredients.

In one embodiment, the disclosed compositions comprise from about 15% to about 30% by weight of ingredients chosen from:
a) from about 10% to about 90% by weight of one or more emulsifiers and antistatic agents;
   i) from about 35% to about 60% by weight of one or more antistatic agents having the following formula:

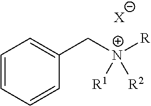

wherein R is C₁₀-C₂₄ linear or branched alkyl, R¹ and R² are each independently methyl, ethyl, or benzyl, X is a keratin-containing fiber compatible anion;
ii) from about 40% to about 65% by weight of one or more emulsifiers chosen from:
   a) from about 5% to about 30% by weight of one or more C₁₀-C₂₄ linear or branched alcohols;
   b) from about 5% to about 30% by weight of one or more dialkyl phosphates having the formula:

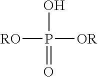

wherein R is C₁₀-C₂₄ linear or branched alkyl; and
c) from about 5% to about 30% by weight of one or more C₁₀-C₂₄ linear or branched mono- or di-polyoxyethylene alkyl phosphates having the formula:

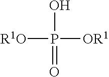

wherein each R¹ is independently hydrogen or a unit having the formula:

—(CH₂CH₂O)ₙR²
R² is C₁₀-C₂₄ linear or branched alkyl and the index n is from about 2 to about 20; and
d) from about 0.55% to about 10% by weight of one or more quaternary ammonium compounds having the formula:

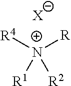

wherein R is C₁₀-C₂₄ linear or branched alkyl, R¹, R² and R³ are each independently C₁-C₃ linear alkyl, X is a keratin-containing fiber compatible anion; and
b) the balance emollients, wetting agents, preservatives, antimicrobials and one or more carriers.

In one iteration of this embodiment, the disclosed compositions comprise from about 15% to about 30% by weight of ingredients chosen from:
a) from about 10% to about 90% by weight of one or more active ingredients chosen from:
   i) from about 35% to about 60% by weight of benzyldimethylocta-decylazanium chloride;
   ii) from about 15% to about 35% by weight of an admixture of 1-hexadecanol and 1-octadecanol;
   iii) from about 15% to about 30% by weight of an admixture of 1-hexadecanol, 1-octadecanol and dihexadecanylphosphate, and an admixture of PEG-10 hexadecanyl/oleyl monophosphate and diphosphate; and
   iv) from about 0.5% to about 5% by weight of trimethylhexadecanyl-ammonium chloride; and
b) the balance emollients, wetting agents, preservatives, antimicrobials, opacifiers, pH balancers, and one or more carriers.

In another embodiment, the disclosed compositions comprise from about 15% to about 30% by weight of ingredients chosen from:
a) from about 10% to about 90% by weight of one or more emulsifiers and antistatic agents;
b) from about 10% to about 25% by weight of one or more active ingredients chosen from:
   i) from about 10% to about 25% by weight of one or more wetting agents;
   ii) from about 10% to about 25% by weight of one or more emollients;
   iii) from about 5% to about 15% by weight of one or more antimicrobial agents;
iv) from about 25% to about 45% by weight of one or more preservatives; and
   v) from about 1% to about 7% by weight of one or more pH balancers;
c) the balance one or more carriers.

In one iteration of this embodiment, the composition comprises from about 10% to about 25% by weight of one or more active ingredients comprise:
i) from about 10% to about 25% by weight of one or more wetting agents chosen from polyalkyl siloxanes, polyaryl siloxanes, polyalkylaryl siloxanes, polyether siloxane copolymers, amino substituted silicones, quaternized silicones, and mixtures thereof;
ii) from about 10% to about 25% by weight of one or more emollients chosen from C₃-C₈ hydroxyalkyl mono- and di-N-hydroxyethyl amides, C₁₄-C₂₂ fatty alcohols, C₂₂ fatty alcohols fatty acid esters, C₈-C₄₀ hydrocarbons, and mixtures thereof;
iii) from about 5% to about 15% by weight of one or more antimicrobial agents chosen from 1,3-bis(hydroxymethyl)-5,5-dimethylimidazolidine-2,4-dione, antimicrobials as disclosed in U.S. Pat. No. 4,329,336, included herein by reference in its entirety, 1-imidazolyl-1-(4-chlorophenoxy)-3,3-dimethylbutan-2-one;
iv) from about 25% to about 45% by weight of one or more preservatives; and
v) from about 1% to about 7% by weight of one or more pH balancers.

### Phase A

Phase A of the disclosed cleansing compositions comprises a stabilized carrier system. In one embodiment, Phase A comprises water comprising from about 0.01% to about 2% by weight of one or more stabilizers or thickeners. The following are non-limiting examples of Phase A suitable for use in the disclosed cleansing compositions.

TABLE I

| Ingredients | A1 | A2 | A3 | A4 | A5 |
|---|---|---|---|---|---|
| Guar gum, 2-hydroxy-3- | 0.131 | 0.133 | 0.128 | 0.193 | 0.207 |
| (trimethylammonio)- | | | | | |
| propylether chloride | | | | | |
| water | balance | balance | balance | balance | balance |

### Phase B

Phase B comprises emollients, wetting agents, conditioners, moisturizers, and the like, as well as emulsion stabilizers, preservatives and the like that when taken together with Phase A provides a stable, flowable base for delivering the keratin-containing fiber restorative ingredients of Phase C while simultaneously providing the keratin-containing fibers with a soft, smooth, non-static combable texture. The ingredients of Phase B can trap and help remove the accumulated by-products caused by chemical, environmental, and mechanical damage to keratin-containing fibers. The following are non-limiting examples of Phase B suitable for use in the disclosed cleansing compositions.

TABLE II

| Ingredients | B1 | B2 | B3 | B4 | B5 |
|---|---|---|---|---|---|
| Triethanolamine | 0.47 | 0.4 | 0.5 | 0.4 | 0.4 |
| 2-hydroxy-N-(2-hydroxyethyl)- | 2.34 | 2.7 | 2.4 | 2.7 | 2 |
| propanamide | | | | | |
| PEG-12 dimethicone | 2.34 | 2.5 | 2.6 | 2.7 | 2 |
| Cetearyl alcohol dicetyl | 21.1 | 20.4 | 21.7 | 20 | 26.3 |
| phosphate | | | | | |
| ceteth-10 phosphate | | | | | |
| Cetearyl Alcohol | 18.7 | 20.4 | 17.3 | 22.1 | 17.5 |
| imidazolidinyl urea | 2.3 | 2.3 | 2.2 | 2.2 | 2.2 |
| phenoxyethanol | 2.3 | 2.3 | 2.2 | 2.2 | 2.2 |
| stearalkonium chloride | 37.4 | 36.3 | 39 | 35.4 | 35.1 |
| glycol distearate | 9.3 | 9 | 8.7 | 8.8 | 8.8 |
| cetyl trimethyl ammonium | 2.34 | 2.3 | 2.1 | 2.2 | 2.2 |
| chloride | | | | | |
| 1,3-bis(hydroxymethyl)-5,5- | 1.41 | 1.4 | 1.3 | 1.3 | 1.3 |
| dimethylimidazolidine-2,4- | | | | | |
| dione | | | | | |

### Phase C

Phase C comprises keratin-containing fiber restorative ingredients which penetrate the damaged hair cuticle and are absorbed into the cortex wherein they are incorporated into the fibers themselves thereby replacing damaged or destroyed portions of the keratin.

TABLE III

| Ingredients | C1 | C2 | C3 | C4 | C5 |
|---|---|---|---|---|---|
| Safflower oleosomes | 42 | 44 | 36.5 | 46.5 | 41.3 |
| colorant | 8.4 | 8 | 9.1 | 7.7 | 8.2 |
| Fragrance | 42 | 40 | 45.7 | 38.8 | 41.3 |
| Cholesterol | 2.1 | 2.4 | 2.7 | 1.9 | 2.9 |
| Olive oil | 2.1 | 2.4 | 2.3 | 1.9 | 2.9 |
| cystine bis-PG-propyl | 0.4 | 0.4 | 0.5 | 0.39 | 0.4 |
| silanetriol | | | | | |
| helianths annuus seed oil | 2.1 | 1.9 | 2.3 | 1.9 | 2 |
| and caviar exract | | | | | |
| Prodew ™ 500 | 0.004 | 0.004 | 0.004 | 0.004 | 0.004 |
| Photoprotectant | 0.4 | 0.4 | 0.4 | 0.39 | 0.4 |
| β-cyclodextrin | 0.4 | 0.4 | 0.4 | 0.39 | 0.4 |
| Water | balance | balance | balance | balance | balance |

### Examples 1-5

The following are non-limiting examples of compositions useful for treating damaged keratin-containing fibers. Examples 1-5 were prepared by the general procedure as follows. The disclosed examples can be used on any type of keratin-containing fiber, especially human hair, either by a single user or applied by a professional, i.e., a stylist. The restorative effects produced on keratin-containing fibers are reproducible, as well as cumulative when applied as necessary or directed.

Phase A was prepared by charging a reaction vessel with water (10 L) and slowly adding and dissolving Guar gum, 2-hydroxy-3-(trimethylammonio)propylether chloride (13.13 g) to form Phase A1 as disclosed in Table I. Once dissolved, the solution was heated to from 70° C. to about 80° C. and the components of Phase B as exemplified in Example B1 are added in the following order and amounts as listed in Table IV with efficient mixing. The amounts given in Table IV are the amounts of the listed trade name products and, as such, each ingredient can include water that is present as a carrier or as part of a hydrate.

TABLE IV

| | | Amount |
|---|---|---|
| Ingredients | Trade name | (g) |
| Triethanolamine | — | 13.1 |
| 2-hydroxy-N-(2-hydroxyethyl)- | Mackamide ™ LME | 65.7 |
| propanamide | | |
| PEG-12 dimethicone | DC-193 Surfactant | 65.7 |
| cetearyl alcohol, dicetyl phosphate, | Crodafos ™ CES | 590.9 |
| ceteth-10 phosphate admixture | | |
| Cetearyl Alcohol | Crodacol ™ 1618 | 525.2 |
| imidazolidinyl urea | Germall ™ 115 | 65.7 |
| phenoxyethanol | Sepicide ™ LD | 65.7 |
| stearalkonium chloride | Mackernium ™ SDC-85 | 1.50.4 |
| glycol distearate | Mackester ™ EGDS | 262.6 |
| cetyl trimethyl ammonium chloride | Jeequat ™ CT-29 | 65.7 |
| 1,3-bis(hydroxymethyl)-5,5- | Mackstart ™ DM | 39.4 |
| dimethylimidazolidine-2,4-dione | | |

Following the addition of 1,3-bis(hydroxymethyl)-5,5-dimethylimidazolidine-2,4-dione (DMDM hydantoin) mixing was continued for an additional 30 minutes after which the solution was cooled to approximately 45° C. to 50° C. Once cooled the components of Phase C as exemplified in Example C1 are added in the following order and amounts shown in Table V.

TABLE V

| Ingredients | Trade name | Amount (g) |
|---|---|---|
| Safflower oleosomes | Hydresia ™ G-2 | 131.3 |
| Colorant | Covapearl ™ Silver | 26.3 |
| | 939AS | |
| Fragrance | — | 131.2 |
| Cholesterol | Fancol ™ CH | 6.6 |
| Olive oil | Fancor/Jeen | 6.6 |
| cystine bis-PG-propyl silanetriol | Crodasone ™ Cysteine | 1.3 |
| helianths annuus seed oil and | Lipidami ™ Caviar | 6.6 |
| caviar exract | | |
| Prodew ™ 500 | Ajinomoto | 0.013 |
| Photoprotectant | Nano-Lipobelle ™ UV- | 1.3 |
| | A/B | |
| β-cyclodextrin | Caramax ™ W-7 | 1.3 |

After the addition of β-cyclodextrin was complete the admixture was stirred while cooling to about 30° C. until the solution appeared to be completely homogeneous. The resulting composition was a flowable white pearlized cream with a specific gravity of 0.96 g/mL and pH of 3.75. In a like manner, Phase A2 (75.1 wgt. equiv.), Phase B2 (22.05 wgt. equiv.) and Phase C2 (2.5 wgt. equiv.) are combined to produce the composition of Example 2. In a similar like manner, Phase A3 (72.6 wgt. equiv.), Phase B3 (23.07 wgt. equiv.) and Phase C3 (2.19 wgt. equiv.) are combined to produce the composition of Example 3. Similarly, Phase A4 (77.75 wgt. equiv.), Phase B4 (22.6 wgt. equiv.) and Phase C4 (2.58 wgt. equiv.) are combined to produce the composition of Example 4. As describe above, Phase A5 (72.35 wgt. equiv.), Phase B5 (22.8 wgt. equiv.) and Phase C5 (2.42 wgt. equiv.) are combined to produce the composition of Example 5. The resulting compositions Examples 1-5 comprise the following ingredients in the following approximate amounts on a % weight/weight basis.

TABLE VI

| Ingredients | 1 | 2 | 3 | 4 | 5 |
|---|---|---|---|---|---|
| guar hydroxypropyl | 0.1 | 0.1 | 0.1 | 0.15 | 0.15 |
| triammonium chloride | | | | | |
| Triethanolamine | 0.1 | 0.1 | 0.12 | 0.1 | 0.1 |
| 2-hydroxy-N-(2- | 0.5 | 0.6 | 0.55 | 0.6 | 0.45 |
| hydroxyethyl)- | | | | | |
| propanamide | | | | | |
| PEG-12 dimethicone | 0.5 | 0.55 | 0.6 | 0.6 | 0.45 |
| Cetearyl alcohol dicetyl | 4.5 | 45 | 5 | 4.5 | 6 |
| phosphate | | | | | |
| ceteth-10 phosphate | | | | | |
| Cetearyl Alcohol | 4 | 4.5 | 4 | 5 | 4 |
| imidazolidinyl urea | 0.5 | 0.5 | 0.5 | 0.5 | 0.5 |
| phenoxyethanol | 0.5 | 0.5 | 0.5 | 0.5 | 0.5 |
| stearalkonium chloride | 8 | 8 | 9 | 8 | 8 |
| glycol distearate | 2 | 2 | 2 | 2 | 2 |
| cetyl trimethyl | 0.5 | 0.5 | 0.5 | 0.5 | 0.5 |
| ammonium chloride | | | | | |
| 1,3-bis(hydroxymethyl)- | 0.3 | 0.3 | 0.3 | 0.3 | 0.3 |
| 5,5-dimethylimidazol- | | | | | |
| idine-2,4-dione | | | | | |
| Safflower oleosomes | 1 | 1.1 | 0.8 | 1.2 | 1 |
| Colorant | 0.2 | 0.2 | 0.2 | 0.2 | 0.2 |
| Fragrance | 1 | 1 | 1 | 1 | 1 |
| Cholesterol | 0.05 | 0.06 | 0.06 | 0.05 | 0.07 |
| Olive oil | 0.05 | 0.06 | 0.05 | 0.05 | 0.07 |
| cystine bis-PG-propyl | 0.01 | 0.01 | 0.01 | 0.01 | 0.01 |
| silanetriol | | | | | |
| helianths annuus seed oil | 0.05 | 0.05 | 0.05 | 0.05 | 0.05 |
| and caviar exract | | | | | |
| Prodew ™ 500 | 0.0001 | 0.0001 | 0.0001 | 0.0001 | 0.0001 |
| Photoprotectant | 0.01 | 0.01 | 0.01 | 0.01 | 0.01 |
| β-cyclodextrin | 0.01 | 0.01 | 0.01 | 0.01 | 0.01 |
| water | balance | balance | balance | balance | balance |

### Restructuring and Repair Compositions

Another aspect of the disclosed compositions relates to treatments that can be used before and/or after applying an oxidative dyeing composition to keratin-containing fibers. The compositions of this aspect provide restructuring and repair of damage fiber protein and thereby assist in the natural restructuring and repair process. In addition, the composition delivers ingredients which provide an enhanced aesthetic appearance to the fibers, for example, compounds which provide a desirable luster to hair. The compositions can further serve to affect the bulk properties of the fibers themselves; by lowering the residual fiber pH which can increase as a direct result of oxidative dyeing and to correct the outer layer porosity.

The compositions comprise:
A) from about 0.1% to about 5% by weight of a protein restorative system;
B) from about 1% to about 15% by weight of a keratin-containing fiber conditioning and moisturizing fiber protectant system; and
C) the balance composition stabilizers, aesthetics and carriers, and the like.

The restorative and repair compositions provide the necessary admixture of amino acids and/or sources of amino acids such that these critical ingredients can be taken up by the keratin-containing fibers. For example, the human body cannot synthesize the amino acids lysine and arginine, and, as such, these amino acids must be obtained from an outside source. It is known that the amino acid L-lysine inhibits the enzyme alopecia which is responsible for baldness whether hereditary, environmental or due to a hormonal imbalance. L-Arginine on the other hand, serves as a biological precursor of nitric oxide, which is a messenger substance that stimulates hair growth by opening potassium channels. Therefore, compositions comprising these two amino acids can positively affect the rate at which keratin-containing fiber can be restored by the body.

A first embodiment of the disclosed restructuring and repair compositions, comprise from about from about 0.1% to about 5% by weight of a protein restorative system, comprising:
a) from about 25% to about 50% by weight of a source of lysine;
b) from about 25% to about 50% by weight of a source of glycine;
c) from about 7% to about 22% by weight of a source of arginine; and
d) from about 1% to about 15% by weight of a source of cysteine.

In one iteration of this embodiment, the compositions comprise:
a) from about 35% to about 45% by weight of lysine or a salt thereof;
b) from about 35% to about 45% by weight of glycine or a salt thereof;
c) from about 10% to about 20% by weight of arginine or a salt thereof; and
d) from about 5% to about 10% by weight of one or more sources of cysteine chosen from hydrolyzed protein, enzymatically reduced protein, the amino acid cysteine, sources of cystine, and mixtures thereof.

Another embodiment of the disclosed restructuring and repair compositions, comprise from about from about 1% to about 15% by weight of a keratin-containing fiber conditioning and moisturizing fiber protectant system, comprising:
a) from about 1% to about 15% by weight of one or more moisturizers;
b) from about 1% to about 15% by weight of one or more wetting agents;
c) from about 40% to about 75% by weight of one or more emollients;
d) from about 30% to about 45% by weight of one or more humectants;
e) from about 0.5% to about 3% by weight of one or more surfactants; and
f) from about 0.5% to about 3% by weight of one or more conditioning agents.

In one embodiment, the compositions comprise from about 3% to about 10% by weight of a protein restorative system, comprising:
a) from about 5% to about 10% by weight of one or more moisturizers;
b) from about 5% to about 10% by weight of one or more wetting agents;
c) from about 45% to about 60% by weight of one or more emollients;
d) from about 33% to about 40% by weight of one or more humecticants;
e) from about 1% to about 3% by weight of one or more surfactants; and
f) from about 1% to about 3% by weight of one or more conditioning agents.

The balance of the restorative and repair compositions comprise stabilizers, aesthetics and carriers. In one embodiment, water is the carrier. In another embodiment, the compositions comprises and admixture of water and from about 5% to about 20% by weight of the composition one or more alcohols. Non-limiting examples of suitable alcohols include ethanol, n-propanol, iso-propanol, and propanediol. The formulator can, however, choose any mixture of organic solvents and/or water in combination which affords a stabile and homogeneous solution.

### Phase A

Phase A comprises a stabilized aqueous based carrier containing one or more emollients, surfactants moisturizers, anti-oxidants, pH control agents, chelants, humectants, amino acids, sources of amino acids, and the like. Non-limiting examples of Phase A compositions are described in Table VII. Unless otherwise indicated, the amounts in the following tables are weight % of the total Phase.

TABLE VII

| Ingredients | A1 | A2 | A3 | A4 | A5 |
|---|---|---|---|---|---|
| Propanediol | 12.3 | 13 | 12.6 | 11.8 | 12.1 |
| glycerine, glyceryl | 0.5 | 0.7 | 0.5 | 0.5 | 0.7 |
| acrylate/acrylic acid | | | | | |
| copolymer, propylene | | | | | |
| glycol admixture | | | | | |
| 1,3-bis(hydroxymethyl)- | 0.5 | 0.5 | 0.5 | 0.5 | 0.5 |
| 5,5-dimethylimidazol- | | | | | |
| idine-2,4-dione | | | | | |
| PEG-12 dimethicone | 0.5 | 0.55 | 0.6 | 0.5 | 0.5 |
| dimethicone PEG-8 | 1.5 | 1.4 | 1.55 | 1.6 | 1.4 |
| Meadowfoamate | | | | | |
| lysine hydrochloride | 0.5 | 0.5 | 0.5 | 0.5 | 0.5 |
| sodium citrate | 2 | 2 | 2 | 2 | 2.1 |
| arginine | 0.2 | 0.2 | 0.2 | 0.2 | 0.2 |
| α-lipoic acid | 0.05 | 0.05 | 0.05 | 0.05 | 0.05 |
| imidazolidinyl urea | 0.5 | 0.5 | 0.5 | 0.5 | 0.5 |
| β-cyclodextrin | 0.2 | 0.5 | 0.5 | 0.5 | 0.5 |
| Glycine | 0.5 | 0.5 | 0.5 | 0.5 | 0.5 |
| EDTA | 1.5 | 1.5 | 1.5 | 1.5 | 1.5 |
| 2-hydroxy-N- | 2.5 | 3.1 | 2.75 | 2.5 | 2.25 |
| (2-hydroxyethyl)- | | | | | |
| propanamide | | | | | |
| water | balance | balance | balance | balance | balance |

### Phase B

Phase B can comprise one or more aesthetic ingredients, preservatives, and the like that is combined with a one or more emollients. Non-limiting examples of Phase B are disclosed in Table VII.

TABLE VIII

| Ingredients | B1 | B2 | B3 | B4 | B5 |
|---|---|---|---|---|---|
| Fragrance-1 | 16.7 | — | — | 16.7 | — |
| Fragrance-2 | — | 16.7 | — | — | 16.7 |
| Fragrance-3 | — | — | 16.7 | — | — |
| PPG-5-Ceteth-20 | 66.6 | 66.6 | 66.6 | — | — |
| PLURONIC ™ F68 | — | — | — | 66.6 | 66.6 |
| Phenoxyethanol | 16.7 | 16.7 | 16.7 | 16.7 | 16.7 |

### Phase C

Phase C comprises amino acids, sources of amino acids, antimicrobial agents, natural surfactants, condition agents, aesthetics, and the like. Non-limiting examples of Phase C are disclosed in Table IX.

TABLE IX

| Ingredients | C1 | C2 | C3 | C4 | C5 |
|---|---|---|---|---|---|
| Hydrolyzed Keratein | 43.5 | 44.4 | 48 | 37.5 | 42 |
| Gynostemma Pentapphyllum | 8.7 | 9.7 | 8 | 8.3 | 10.1 |
| Extract, Panax Ginseng | | | | | |
| Extract | | | | | |
| Cystine Bis-PG-Propyl | 8.7 | 9.7 | 8 | 16.7 | 101 |
| Silanetriol | | | | | |
| Photoprotectant | 8.7 | 8.1 | 8 | 8.3 | 8.4 |
| Helianthus | 4.3 | 4 | 4 | 4.2 | 4.2 |
| Annuus(Sunflower)Seed Oil | | | | | |
| and Caviar Extract | | | | | |
| Colorant | 26.1 | 24.1 | 24 | 25 | 25.2 |

### Examples 6-10

The following are non-limiting examples of compositions useful for restoring and repairing keratin-containing fibers. Examples 6-10 were prepared by the general procedure as follows. The disclosed examples can be used on any type of keratin-containing fiber, especially human hair, either by a single user or applied by a professional, i.e., a stylist. The restorative effects produced on keratin-containing fibers are reproducible, as well as cumulative when applied as necessary or directed.

Phase A was prepared by charging a reaction vessel with water (10,000 g) and adding the ingredients disclosed in Table X. The admixture is heated to about 70° C. and stirred until homogeneous thereby forming Phase A1 as disclosed in Table VII.

TABLE X

| Ingredients | Trade name | Amount (g) |
|---|---|---|
| Propanediol | Zemea ™ propanediol | 1604.3 |
| glycerine, glyceryl acrylate/acrylic | Lubrajel ™ CG | 66.8 |
| acid copolymer, propylene glycol | | |
| admixture | | |
| 1,3-bis(hydroxymethyl)-5,5- | Mackstart ™ DM | 66.8 |
| dimethylimidazolidine-2,4-dione | | |
| PEG-12 dimethicone | DC-193 Surfactant ™ | 66.8 |
| dimethicone PEG-8 | Fancorsil ™ Lim-1 | 20.5 |
| meadowfoamate | | |
| lysine hydrochloride | — | 66.8 |
| sodium citrate | — | 276.4 |
| arginine | — | 26.7 |
| α-lipoic acid | — | 6.7 |
| imidazolidinyl urea | Germall ™ 115 | 66.8 |
| β-cyclodextrin | Caramax ™ W-7 | 26.7 |
| Glycine | — | 66.8 |
| EDTA | Dissolvin ™ Z | 200.5 |
| 2-hydroxy-N-(2-hydroxyethyl)- | Mackamide ™ LME | 334.2 |
| propanamide | | |
| water | — | 10,000 |

Once the ingredients that comprise Phase A were combined the solution was stirred at about 70° C. to 75° C. for an additional 30 minutes then cooled to about 45° C. to 50° C. Phase B as disclosed in Table XI corresponding to B1 of Table VIII was pre-combined then added at once to the stirred solution.

TABLE XI

| Ingredients | Trade name | Amount (g) |
|---|---|---|
| fragrance | — | 66.8 |
| PPG-5-Ceteth-20 | Procetyl ™ AWS | 267.4 |
| phenoxyethanol | Sepicide ™ LD | 66.8 |

After the addition of Phase B, the solution was stirred an addition 10 minutes. While cooling to 30° C., the ingredients disclosed in Table XII are added in order. This corresponds to Phase C1 disclosed in Table IX herein above.

TABLE XII

| Ingredients | Trade name | Amount (g) |
|---|---|---|
| Hydrolyzed Keratein | Kera-Tein ™ 1000 | 66.8 |
| Gynostemma Pentapphyllum | Actigen ™ O2 GL | 13. |
| Extract, Panax Ginseng Extract | | |
| Cystine Bis-PG-Propyl Silanetriol | Crodasone ™ Cysteine | 13.4 |
| Photoprotectant | Covabsorb ™ EW | 13.4 |
| Helianthus | Lipidami ™ Caviar | 6.7 |
| Annuus(Sunflower)Seed Oil and | | |
| Caviar Extract | | |
| colorant | Covapearl ™ Silver | 40.1 |
| | 939AS | |

The resulting composition is a pearlized white liquid with a specific gravity of 1.037 g/mL and pH of 4.24. In a like manner, Phase A2 (97.2 equiv.), Phase B2 (3 equiv.) and Phase C2 (1.24 equiv.) are combined to produce the composition of Example 7. In a similar like manner, Phase A3 (99.15 equiv.), Phase B3 (3 equiv.) and Phase C3 (1.25 equiv.) are combined to produce the composition of Example 8. Similarly, Phase A4 (97.85 equiv.), Phase B4 (3 equiv.) and Phase C4 (1.2 equiv.) are combined to produce the composition of Example 4. As describe above, Phase A5 (97.45 equiv.), Phase B5 (3 equiv.) and Phase C5 (1.19 equiv.) are combined to produce the composition of Example 5. The resulting compositions Examples 6-10 comprise the following ingredients in the following approximate amounts on a % weight/weight basis.

TABLE VI

| Ingredients | 6 | 7 | 8 | 9 | 10 |
|---|---|---|---|---|---|
| Propanediol | 12 | 13 | 12.5 | 11.5 | 11.75 |
| glycerine, glyceryl | 0.5 | 0.7 | 0.5 | 0.5 | 0.7 |
| acrylate/acrylic acid | | | | | |
| copolymer, propylene | | | | | |
| glycol admixture | | | | | |
| 1,3-bis(hydroxymethyl)- | 0.5 | 0.5 | 0.5 | 0.5 | 0.5 |
| 5,5-dimethylimidazol- | | | | | |
| idine-2,4-dione | | | | | |
| PEG-12 dimethicone | 0.5 | 0.55 | 0.6 | 0.5 | 0.5 |
| dimethicone PEG-8 | 1.5 | 1.4 | 1.55 | 1.6 | 1.4 |
| Meadowfoamate | | | | | |
| lysine hydrochloride | 0.5 | 0.5 | 0.5 | 0.5 | 0.5 |
| sodium citrate | 2 | 2 | 2 | 2 | 2 |
| arginine | 0.2 | 0.2 | 0.2 | 0.2 | 0.2 |
| α-lipoic acid | 0.05 | 0.05 | 0.05 | 0.05 | 0.05 |
| imidazolidinyl urea | 0.5 | 0.5 | 0.5 | 0.5 | 0.5 |
| β-cyclodextrin | 0.2 | 0.5 | 0.5 | 0.5 | 0.5 |
| Glycine | 0.5 | 0.5 | 0.5 | 0.5 | 0.5 |
| EDTA | 1.5 | 1.5 | 1.5 | 1.5 | 1.5 |
| 2-hydroxy-N- | 2.5 | 3.1 | 2.75 | 2.5 | 2.25 |
| (2-hydroxyethyl)- | | | | | |
| propanamide | | | | | |
| fragrance | 0.5 | 0.5 | 0.5 | 0.5 | 0.5 |
| PPG-5-Ceteth-20 | 2 | 2 | 2 | 2 | 2 |
| Phenoxyethanol | 0.5 | 0.5 | 0.5 | 0.5 | 0.5 |
| Hydrolyzed Keratein | 0.5 | 0.55 | 0.6 | 0.45 | 0.5 |
| Gynostemma | 0.1 | 0.1 | 0.1 | 0.1 | 0.12 |
| Pentapphyllum Extract, | | | | | |
| Panax Ginseng Extract | | | | | |
| Cystine Bis-PG-Propyl | 0.1 | 0.12 | 0.1 | 0.2 | 0.12 |
| Silanetriol | | | | | |
| Photoprotectant | 0.1 | 0.1 | 0.1 | 0.1 | 0.1 |
| Helianthus | 0.05 | 0.05 | 0.05 | 0.05 | 0.05 |
| Annuus(Sunflower)Seed | | | | | |
| Oil and Caviar Extract | | | | | |
| Colorant | 0.3 | 0.3 | 0.3 | 0.3 | 0.3 |
| water | balance | balance | balance | balance | balance |

### Methods

Disclosed herein are methods for protecting, restoring, and/or repairing keratin-containing fibers, comprising contacting keratin-containing fibers with one or more of the disclosed compositions.

### Kits

Disclosed herein are kits for protecting, restoring, and/or repairing keratin-containing fibers. One aspect of the disclosed kits contains a composition comprising:
A) from about 70% to about 80% by weight of a stabilized carrier system, comprising:
   a) from about 0.01% to about 5% by weight of one or more aqueous solution stabilizers or thickeners; and
   b) the balance one or more carriers;
B) from about 15% to about 35% by weight of a keratin-containing fiber conditioning and moisturizing protectant system, comprising:
   a) from about 50% to about 90% by weight of one or more active ingredients chosen from:
      i) optionally from about 25% to about 60% by weight of one or more anti-static agents;
      ii) optionally from about 5% to about 30% by weight of one or more emulsion stabilizers;
      iii) optionally from about 5% to about 90% by weight of one or more emulsifiers; and
      iv) optionally from about 1% to about 15% by weight of one or more fiber surface protection agents chosen from conditioners, wetting agents, rinse aids, emollients and mixtures thereof;
   b) from about 01% to about 25% by weight of one or more preservatives, bactericidal, bacteriostatic compounds, and mixtures thereof; and
   c) from about 0.05% to about 5% by weight of one or more chelants, pH balancers, and carriers;
C) from about 0.5% to about 10% by weight of one or more keratin-containing fiber restorative, keratin-containing fiber protective ingredients and mixtures thereof; and
D) the balance carriers and adjunct ingredients.

Another aspect of the disclosed kits contains a composition comprising:
A) from about 0.1% to about 5% by weight of a protein restorative system, comprising:
   i) one or more essential amino acids wherein the term essential amino acid refers to amino acids that cannot be synthesized by a human; and
   ii) optionally one or more non-essential amino acids;
B) from about 1% to about 15% by weight of a keratin-containing fiber conditioning and moisturizing fiber protectant system, comprising one or more optionally present ingredients chosen from each of the following: moisturizers, wetting agents, emollients, humectants, surfactants ad conditioning agents; and
C) the balance, comprising:
   i) optionally from about 1% to about 10% by weight of the balance, one or more composition stabilizers, aesthetics, UV protectants, protein enrichment agents, chelants, antioxidants, preservatives, pH control agents, perfumes, colorants and the like; and
   ii) one or more carriers.

The disclosed kits can further comprise one or more containers, inter alia, bottles, spray bottles, pouches, and the like as known to the artisan. In addition, the kits can comprise instructions for use or instructions for determining the method of use based upon described criteria. This optional criteria can be used by a subject, i.e., consumer or the professional as a means to determine the effective amount of the disclosed composition to be used. In addition, the criteria can contain metrics for determining the level of effectiveness and, as such, for example, the amount to of product to continue to use, a reduced amount to use, or whether the use can be discontinued.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A composition comprising:
A) from about 70% to about 80% by weight of a stabilized carrier system, comprising:
a) from about 0.01% to about 5% by weight of one or more aqueous solution stabilizers or thickeners; and
b) the balance one or more carriers;
B) from about 15% to about 35% by weight of a keratin-containing fiber conditioning and moisturizing protectant system, comprising:
a) from about 50% to about 90% by weight of one or more active ingredients chosen from:
i) optionally from about 25% to about 60% by weight of one or more anti-static agents;
ii) optionally from about 5% to about 30% by weight of one or more emulsion stabilizers;
iii) optionally from about 5% to about 90% by weight of one or more emulsifiers; and
iv) optionally from about 1% to about 15% by weight of one or more fiber surface protection agents chosen from conditioners, wetting agents, rinse aids, emollients and mixtures thereof;
b) from about 01% to about 25% by weight of one or more preservatives, bactericidal, bacteriostatic compounds, and mixtures thereof; and
c) from about 0.05% to about 5% by weight of one or more chelants, pH balancers, and carriers;
C) from about 0.5% to about 10% by weight of one or more keratin-containing fiber restorative, keratin-containing fiber protective ingredients and mixtures thereof; and
D) the balance carriers and adjunct ingredients.

2. The composition according to claim 1, wherein the conditioning and moisturizing protectant system, comprises from about 15% to about 30% by weight of ingredients chosen from:
a) from about 10% to about 90% by weight of one or more emulsifiers and antistatic agents;
i) from about 35% to about 60% by weight of one or more antistatic agents having the following formula: 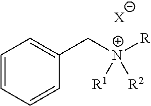
wherein R is C₁₀-C₂₄ linear or branched alkyl, R¹ and R² are each independently methyl, ethyl, or benzyl, X is a keratin-containing fiber compatible anion;
ii) from about 40% to about 65% by weight of one or more emulsifiers chosen from:
a) from about 5% to about 30% by weight of one or more C₁₀-C₂₄ linear or branched alcohols;
b) from about 5% to about 30% by weight of one or more dialkyl phosphates having the formula: 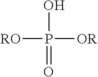
wherein R is C₁₀-C₂₄ linear or branched alkyl; and
c) from about 5% to about 30% by weight of one or more C₁₀-C₂₄ linear or branched mono- or di-polyoxyethylene alkyl phosphates having the formula: 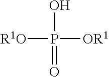
wherein each R¹ is independently hydrogen or a unit having the formula:
—(CH₂CH₂O)ₙR²
R² is C₁₀-C₂₄ linear or branched alkyl and the index n is from about 2 to about 20; and
d) from about 0.55% to about 10% by weight of one or more quaternary ammonium compounds having the formula: 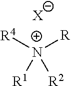
wherein R is C₁₀-C₂₄ linear or branched alkyl, R¹, R² and R³ are each independently C₁-C₃ linear alkyl, X is a keratin-containing fiber compatible anion; and
b) the balance emollients, wetting agents, preservatives, antimicrobials and one or more carriers

3. The composition according to claim 2, comprising from about 10% to about 90% by weight of one or more active ingredients chosen from:
i) from about 35% to about 60% by weight of benzyldimethylocta-decylazanium chloride;
ii) from about 15% to about 35% by weight of an admixture of 1-hexadecanol and 1-octadecanol;
iii) from about 15% to about 30% by weight of an admixture of 1-hexadecanol, 1-octadecanol and dihexadecanylphosphate, and an admixture of PEG-10 hexadecanyl/oleyl monophosphate and diphosphate; and
iv) from about 0.5% to about 5% by weight of trimethylhexadecanyl-ammonium chloride.

4. The composition according to claim 1, comprising from about 4% to about 7% by weight of an admixture of 1-hexadecanol, 1-octadecanol and dihexadecanyl-phosphate, and an admixture of PEG-10 hexadecanyl/oleyl monophosphate and diphosphate.

5. The composition according to claim 1, comprising from about 3% to about 7% by weight of an admixture of 1-hexadecanol and 1-octadecanol

6. The composition according to claim 1, comprising from about 15% to about 30% by weight of ingredients chosen from:
a) from about 10% to about 90% by weight of one or more emulsifiers and antistatic agents;
b) from about 10% to about 25% by weight of one or more active ingredients chosen from:
i) from about 10% to about 25% by weight of one or more wetting agents;
ii) from about 10% to about 25% by weight of one or more emollients;
iii) from about 5% to about 15% by weight of one or more antimicrobial agents;
iv) from about 25% to about 45% by weight of one or more preservatives; and
v) from about 1% to about 7% by weight of one or more pH balancers;
c) the balance one or more carriers.

7. The composition according to claim 6, comprising from about 10% to about 25% by weight of one or more active ingredients comprise:
i) from about 10% to about 25% by weight of one or more wetting agents chosen from polyalkyl siloxanes, polyaryl siloxanes, polyalkylaryl siloxanes, polyether siloxane copolymers, amino substituted silicones, quaternized silicones, and mixtures thereof;
ii) from about 10% to about 25% by weight of one or more emollients chosen from C₃-C₈ hydroxyalkyl mono- and di-N-hydroxyethyl amides, C₁₄-C₂₂ fatty alcohols, C₂₂ fatty alcohols fatty acid esters, C₈-C₄₀ hydrocarbons, and mixtures thereof;
iii) from about 5% to about 15% by weight of one or more antimicrobial agents chosen from 1,3-bis(hydroxymethyl)-5,5-dimethylimidazolidine-2,4-dione, 1-imidazolyl-1-(4-chlorophenoxy)-3,3-dimethylbutan-2-one;
iv) from about 25% to about 45% by weight of one or more preservatives; and
v) from about 1% to about 7% by weight of one or more pH balancers

8. The composition according to claim 1, comprising from about 0.1% to about 2% by weight of a silicone polyethylene glycol copolymer.

9. The composition according to claim 1, comprising from about 0.01% to about 0.1% by weight of an admixture containing the extracts from Helianthus annuus seed oil and caviar.

10. The composition according to claim 1, comprising from about 0.1% to about 2% by weight of one or more emollients chosen from C₃-C₈ hydroxyalkyl mono- and di-N-hydroxyethyl amides.

11. A composition comprising:
A) from about 0.1% to about 5% by weight of a protein restorative system, comprising:
i) one or more essential amino acids wherein the term essential amino acid refers to amino acids that cannot be synthesized by a human; and
ii) optionally one or more non-essential amino acids;
B) from about 1% to about 15% by weight of a keratin-containing fiber conditioning and moisturizing fiber protectant system, comprising one or more optionally present ingredients chosen from each of the following: moisturizers, wetting agents, emollients, humectants, surfactants ad conditioning agents; and
C) the balance, comprising:
i) optionally from about 1% to about 10% by weight of the balance, one or more composition stabilizers, aesthetics, UV protectants, protein enrichment agents, chelants, antioxidants, preservatives, pH control agents, perfumes, colorants and the like; and
ii) one or more carriers.

12. The composition according to claim 11, comprising from about from about 0.1% to about 5% by weight of a protein restorative system, comprising:
a) from about 25% to about 50% by weight of a source of lysine;
b) from about 25% to about 50% by weight of a source of glycine;
c) from about 7% to about 22% by weight of a source of arginine; and
d) from about 1% to about 15% by weight of a source of cysteine.

13. The composition according to claim 12, wherein the source of cysteine is chosen from hydrolyzed protein, enzymatically reduced protein, the amino acid cysteine, sources of cystine, and mixtures thereof.

14. The composition according to claim 13, wherein the source of cysteine is hydrolyzed keratin.

15. The composition according to claim 11, comprising:
a) from about 0.1% to about 1% by weight of lysine, a source of lysine, or a salt thereof
b) from about 0.075% to about 0.8% by weight of arginine, a source of arginine, or a salt thereof;
c) from about 0.1% to about 1% by weight of glycine, a source of glycine, or a salt thereof; and
d) from about 0.1% to about 2% by weight of cysteine, a source of cysteine, or a salt thereof.

16. The composition according to claim 11, comprising from about 1% to about 15% by weight of a keratin-containing fiber conditioning and moisturizing fiber protectant system, comprising:
a) from about 1% to about 15% by weight of one or more moisturizers;
b) from about 1% to about 15% by weight of one or more wetting agents;
c) from about 40% to about 75% by weight of one or more emollients;
d) from about 30% to about 45% by weight of one or more humectants;
e) from about 0.5% to about 3% by weight of one or more surfactants; and
f) from about 0.5% to about 3% by weight of one or more conditioning agents.

17. The composition according to claim 11, comprising from about 0.1% to about 1% by weight of a moisturizing system comprising:
a) from about 10% to about 90% by weight of glycerin;
b) from about 10% to about 90% by weight of glyceryl acrylate/acrylic acid copolymers; and
c) from about 10% to about 90% by weight of propylene glycol.

18. The composition according to claim 11, comprising from about 0.05% to about 0.5% by weight of and admixture of extracts from Gynostemma Pentapphyllum, and Panax Ginseng.

19. The composition according to claim 11, comprising from about 2% to about 3.5% by weight of 2-hydroxy-N-(2-hydroxyethyl)propanamide.

20. The composition according to claim 11, wherein the carrier comprises:
i) from about 5% to about 20% by weight of propanediol; and
ii) from about 80% to about 95% by weight of water.
